(19) United States
(12) Patent Application Publication
(10) Pub. No.: US 2015/0301068 A1
(43) Pub. Date: Oct. 22, 2015

(54) MEANS AND METHOD FOR DIAGNOSIS AND TREATMENT OF ALZHEIMER'S DISEASE

(71) Applicants: VIB VZW, Gent (BE); KATHOLIEKE UNIVERSITEIT LEUVEN, K.U. LEUVEN R&D, Leuven (BE)

(72) Inventors: Bart DE STROOPER, Leuven (BE); Francesc GUIX, Leuven (BE)

(21) Appl. No.: 14/375,426

(22) PCT Filed: Jan. 29, 2013
(86) PCT No.: PCT/EP2013/051682

§ 371 Date: Jul. 29, 2014

Related U.S. Application Data
(60) Provisional application No. 61/592,412, filed on Jan. 30, 2012.

(51) Int. Cl. G01N 33/68; A61K 38/10; C07K 16/28; A61K 38/17; C12Q 1/68; C12N 15/113

(52) U.S. Cl. CPC G01N 33/6896; C12Q 1/6883; C12N 15/1138; C07K 16/28; A61K 38/177; A61K 38/10; C12Q 2600/136; C12Q 2600/158; G01N 2800/2821; C12N 2310/14

(57) ABSTRACT

The disclosure provides an extracellular target for Alzheimer's disease selected from the tetraspanin web family. The disclosure also provides diagnostic methods for the use as a target for detection of Alzheimer's disease in a subject. In addition, screening methods are provided for selecting compounds that bind or down-regulate the expression of the target.

### CROSS-REFERENCE TO RELATED APPLICATIONS

This application is a national phase entry under 35 U.S.C. §371 of International Patent Application PCT/EP2013/051682, filed Jan. 29, 2013, designating the United States of America and published in English as International Patent Publication WO 2013/113696 A1 on Aug. 8, 2013, which claims the benefit under Article 8 of the Patent Cooperation Treaty and under 35 U.S.C. §119(e) to U.S. Provisional Patent Application Ser. No. 61/592,412, filed Jan. 30, 2012.

### TECHNICAL FIELD

The disclosure relates to the field of neurological disorders and, more particularly, to the field of Alzheimer's disease (AD). Specifically, the disclosure provides an extracellular target for Alzheimer's disease selected from the tetraspanin web family. In addition, diagnostic methods are provided for the use of the target for detection of Alzheimer's disease in a subject.

### BACKGROUND

Alzheimer's disease is a progressive neurodegenerative disorder estimated to affect 30 million people worldwide with numbers doubling every 20 years. Alzheimer's disease is characterized by the presence of extraneuronal senile plaques and intraneuronal neurofibrillary tangles (NFT), mainly composed of amyloid beta-peptide (Aβ) and deposits of tau protein, respectively. Although symptoms of Alzheimer's disease manifest early as deficits in memory and other cognitive domains, pathological data show neuropathological features of Alzheimer's disease, including amyloid plaques and neurofibrillary tangles, occur well before the onset of dementia.

Mostly based on studies of families with inherited AD, it is assumed that abnormal Aβ generation is the initial trigger of the disease process (i.e., the amyloid hypothesis) (Hardy and Selkoe, 2002). Aβ is produced when a single type I transmembrane glycoprotein called Amyloid Precursor Protein (APP) is consecutively cleaved by β-secretase and γ-secretase. The steady-state levels of Aβ in the brain are also determined by its clearance via transcytosis through the Blood-Brain Barrier (BBB) and further degradation in the liver (reviewed in Zlokovic, 2008). A fraction of Aβ is also directly degraded in the brain by proteases (reviewed in De Strooper, 2010). Thus, both changes in the production or in the clearance can theoretically cause accumulation of Aβ peptide in the brain.

Amyloid peptides display heterogeneity at their carboxy-terminus, which is readily demonstrated in cell culture and in γ-secretase cell-free assays, suggesting that this heterogeneity is largely generated by the intrinsic properties of the γ-secretase itself (De Strooper et al., 1998, reviewed in De Strooper, 2010). The 40 amino acids length Aβ (Aβ40) is the major form in the brain, while the longer and more neurotoxic form Aβ42 is produced at lower rates by γ-secretase but its presence is pathologically relevant.

There is an unmet need for new biochemical tests that can detect AD disease, and discriminate between AD disease, normal individuals, non-AD disease dementias and other neurological disorders. In addition, there is a need for the identification of novel targets, in particular extracellular targets, as entry points for the development of new medicines for the treatment of AD.

In a previous study carried out by our group directed to study interactors/modulators of the γ-secretase complex, it was discovered that proteins (CD9 and CD81) belonging to the family of the tetraspanins directly interacted with and affected the activity of the complex (Wakabayashi et al., 2009). Tetraspanins are transmembrane proteins that traverse the membrane four times, with conserved charged residues in the transmembrane domains and a defining signature motif in the larger of the two extracellular domains (the EC2). They form associations with other tetraspanins and with other membrane proteins and lipids constituting a specialized type of microdomain: the tetraspanin-enriched microdomain (TEM). TEMs are molecular organizers involved in functions such as membrane trafficking, cell-cell fusion, motility, and signaling. In humans, the tetraspanins form a family of 33 different proteins. We recently investigated if the expression levels of specific tetraspanins change during AD pathology in the brain.

### DISCLOSURE

After checking for the expression of several tetraspanins in the cerebral cortex of healthy individuals and AD patients, we surprisingly found that the expression of tetraspanin 6 (TSPAN6) correlates with the disease stage of Alzheimer's disease. In addition, we found that down-regulation of TSPAN6 in primary neuronal cultures significantly reduced the production of amyloid beta. TSPAN6 is disclosed in the art, for example, in WO2002/012338 where it is used in a screening method for compounds involved in pain, WO2005/026735 discloses that TSPAN6 is differentially expressed in non-steroid dependent cancers, WO2005/064009 teaches the use of TSPAN6 in the classification of cancers, and WO2009/052830 claims the use of a TSPAN6 antibody to treat colorectal cancer, but no reports are disclosed that associate TSPAN6 as a target or as a diagnostic biomarker for Alzheimer's disease.

### BRIEF DESCRIPTION OF THE DRAWINGS

FIG. 1: Representative Western blot showing the increase of both monomer and dimer of TSPAN6 in the prefrontal cortex of the brain during the Braak stages for AD. The protein levels of the protein were quantified from the Western blot shown on the picture, which contains two different samples per Braak stage. From Braak stage 3 on, the protein levels of TSPAN6 increase in a linear way (quantifications of four patients per Braak stage).

FIG. 2: Characterization of the band corresponding to the dimer. (Panel A) Two distinct antibodies against the C-terminus and the N-terminus of the protein were used on a Western blot carried out with lysates (1% TRITON®-X-100) from HEK cells. Both antibodies show the two bands (monomer and dimer). The same two bands are obtained from lysates of HEK cells overexpressing TSPAN6-GFP and using a polyclonal anti-GFP antibody to develop the membrane. (Panel B) The band corresponding to the dimer is not destroyed by any condition: strong detergent (1% SDS), high temperature (95° C.) and presence of a reducer (5% β-mercaptoethanol). This indicates that the nature of the dimer is covalent.

FIG. 3: Localization of TSPAN6 in the mouse brain and during human development. (Panel A) Distinct areas of the mouse brain (White Swiss, 1 year old) were dissected and lysated in 1% TRITON®-X-100 and run in a 4-12% BisTris gel to be later transferred onto a nitrocellulose membrane. Duplicates for each area of the brain were run in parallel (indicated as 1 and 2 on the lanes). TSPAN6 is present in all the areas analyzed. (Panel B) A PCR for TSPAN6 from the total human cDNA obtained from the cerebral cortex of a fetus or an adult. The expression of the mRNA is higher in the fetal brain, indicating a possible important function during development for TSPAN6.

FIG. 4: TSPAN6 is a neuronal protein localized mainly in the axonal processes. (Panel A) Immunofluorescence analysis of fixed rat primary hippocampal neurons fixed with 4% paraformaldehyde and using a polyclonal antibody against TSPAN6. The protein is mainly localized in axons from the very early stages of in vitro development (2 DIV). In mature neurons (10 DIV), it localizes with the presynaptic marker synaptophysin. (Panel B) Western blot from three distinct lysates of primary rat hippocampal neurons or astrocytes. TSPAN6 is present in neurons but not in astrocytes using a polyclonal antibody against TSPAN6 to detect the protein. GFAP and synaptophysin were used as neuronal and astroglial markers, respectively. On the other hand, synaptosomal preparation from an adult rat brain is positive for TSPAN6 as shown in the Western blot at the bottom of the figure.

FIG. 5: TSPAN6 interacts with PS1. (Panel A) Western blot showing the co-immunoprecipitation between TSPAN6 and PS1. HEK cells overexpressing GFP alone or TSPAN6-GFP were lysated and incubated with anti-GFP nanobodies bound covalently to beads. PS1 was detected with a monoclonal anti-PS1 antibody only in the sample containing TSPAN6-GFP. (Panel B) The Western blot of the same lysates does not show any difference in the expression of the components of the γ-secretase complex. There is neither a difference with the complex assembly in the HEK cells overexpressing TSPAN6-GFP, as assessed by Blue Native.

FIG. 6: Down-regulation of TSPAN6 decreases Aβ production. (Panel A) The hamster cell line BHK was transfected with two distinct shRNAs against TSPAN6 and containing an EGFP reporter (lower panel) and the effect on the expression of the protein was assessed by Western blot and compared to non-transfected BHK cells. Both shRNAs were efficient at decreasing the protein levels of TSPAN6. (Panel B) Primary rat hippocampal neurons transfected with a mixture of both shRNAs against TSPAN6 secrete less Aβ into the media after 8 DIV in compare to non-transfected neurons.

FIG. 7: TSPAN6 is secreted in exosomes and is found in the CSF. (Panel A) Western blot of the total lysates or the exosomal fraction of HEK overexpressing GFP alone or TSPAN6-GFP. Only TSPAN6-GFP but not GFP alone is enriched in the exosomal fraction. (Panel B) Western blot of the CSF (25 μL) of two AD patients, showing the presence of TSPAN6.

FIG. 8: Effect of TSPAN6 on Abeta secretion of HEK-APPsw. (Panel A) HEK293-APPsw cells (i.e., HEK293 cells comprising the APP Swedish mutation), 500,000 cells per well seeded on 6-well plates, were transfected with myc-TSPAN6 or left untransfected (control). After 6 hours transfection, the cells' medium was replaced by 0.2% FBS-containing medium. After 24 hours, the medium was collected and the levels of Aβ38, Aβ40 and Aβ42 were determined by ELISA. The overexpression of TSPAN6 increases the levels of Aβ species secreted into the medium. A Western blot was carried out to confirm the ELISA data. 25 μL per sample were run in a 4-12% polyacrylamide gel and transferred onto a nitrocellulose membrane. Epitope retrieval was applied to the samples by boiling the membrane in 1×TBS buffer for 5 minutes. The membrane was incubated with the 6E10 monoclonal antibody against A13. Increased levels of total Aβ was observed in the medium of HEK293 cells overexpressing TSPAN6. (Panel B) In order to determine if the secretion of sAPPα and sAPPβ was altered by the overexpression of TSPAN6, HEK293-APPwt cells (the antibody against sAPPβ only recognizes the wt form) were transfected with myc-TSPAN6 or left untransfected. After 6 hours transfection, the cells medium was replaced by 0.2% FBS-containing medium. After 24 hours, the medium was collected and the levels of sAPPα and sAPPβ was determined by Western blot with a monoclonal 6E10 antibody (SIG-39138, Covance) and a polyclonal anti-sAPPβ antibody (SIG-39138, Covance), respectively.

FIG. 9: Exosome preparation and detection of TSPAN6. (Panel A) TSPAN6 is secreted to the extracellular medium by exosomes. The conditioned media from HEK293 cells untransfected or transfected with Flag-TSPAN6 was collected and proceeded to obtain the exosomal fraction. The total cell lysate and the exosomal fraction from untransfected and transfected HEK293 cells was run in a 4-12% polyacrylamide gel and transferred onto a nitrocellulose membrane. The quality of the exosomes obtained was checked with specific antibodies against calnexin (ER marker, absent in exosomes) and Tsg101 marker (an endosomal protein present in both exosomes and total lysate). Actin and ponceau staining were used as a total protein loading control. A rabbit polyclonal antibody (AP9224b, Abgent) was used to detect TSPAN6. Flag-TSPAN6 and endogenous TSPAN6 was present in exosomes. (Panel B) The same gel was stripped and incubated with an anti-flag antibody to detect overexpressed Flag-TSPAN6 only.

FIG. 10: 25 microL of total CSF samples from AD patients or non-demented subjects were loaded into 4-12% polyacrylamide gels and transferred onto a nitrocellulose membrane. Three different amounts of total protein from HEK293 lysates (1.8 μg, 3.7 μg and 7.5 μg) were loaded in order to make a standard curve for TSPAN6. A rabbit polyclonal antibody (AP9224b, Abgent) was used to detect TSPAN6 (the arrow in the figure indicates TSPAN6). After incubation of the membranes with ECL developing kit during 1 minute, they were developed by exposing them for 30 seconds.

FIG. 11: Comparison of the TSPAN6 levels in the CSF of AD patients (n=16) vs controls (n=16). The intensity of the bands on the membranes containing the CSF samples from AD patients and control subjects of FIG. 10 were quantified with AIDA software. The intensity of the bands was normalized toward the total protein content obtained by ponceau staining. The result of the quantification was normalized with the standard curve made with the total protein from HEK293 cells. The normalized intensity per membrane for the AD samples was compared to that for control subjects in teens of percentage. Student's t-Test was used for statistical analysis.

FIG. 12: Correlation between the levels in the CSF of TSPAN6 and the INNOTEST® Amyloid Tau Index. The relative amount of TSPAN6 in the CSF [R.U] was plotted against the INNOTEST® Amyloid Tau Index (IATI) for those samples where the information was available. Values of IATI<1 was reported for individuals with a typical AD biomarkers profile, whereas values of IATI>1 were found to be typical of healthy control individuals. Most of the individuals with high TSPAN6 content in the CSF show an IATI index<1, whereas those individuals with a low TSPAN6 content in the CSF show an IATI index>1.

FIG. 13: (Panel A) 25 microL of total CSF samples from Lewy Body Dementia (LBD) patients or non-demented subjects were loaded into 4-12% polyacrylamide gels and transferred onto a nitrocellulose membrane. Three different amounts of total protein from HEK293 lysates (2 μg, 4 μg and 8 μs) were loaded in order to make a standard curve for TSPAN6. Ponceau red staining was carried out to obtain the total protein amount per sample. (Panel B) A rabbit polyclonal antibody (AP9224b, Abgent) was used to detect TSPAN6. After incubation of the membranes with ECL developing kit during 1 minute, they were developed by exposing them for 30 seconds.

FIG. 14: Determination of the TSPAN6 levels in patients suffering from Lewy-Body dementia. The intensity of the bands on the membranes containing the CSF samples from LBD patients and control subjects (see FIG. 13) were quantified with AIDA software. The intensity of the bands was normalized toward the total protein content obtained by ponceau staining. The results of the quantification were normalized with the standard curve made with the total protein from HEK293 cells. The normalized intensity per membrane for the LBD samples was compared to that for control subjects in terms of percentage. No differences were observed between control subjects and LBD patients.

FIG. 15: Detection of TSPAN6 in saliva. The saliva sample was collected from a healthy individual who had been one hour without eating or drinking. Immediately after collection, 1× protease inhibitor was added to the sample, which was sonicated at 10× at 10% amplitude and put on ice. Loading buffer containing 5% of β-mercaptoethanol was added into the sample before heating it at 70% for 10 minutes. Finally, a one-minute centrifugation at 14,000 rpm and 4° C. was carried out before loading 40 μl (28 μl sample+12 μl loading buffer) into a 4-12% polyacrylamide gel. After transferring the sample onto a nitrocellulose membrane, it was blotted against a rabbit polyclonal antibody (AP9224b, Abgent) to detect TSPAN6. The arrow points at the presence of TSPAN6 in the saliva sample.

### DETAILED DESCRIPTION

The disclosure provides methods for diagnosing, monitoring and/or staging neurological disorders such as Alzheimer's disease comprising the use of the detection of TSPAN6 in a body sample derived from a patient. The disclosure relates to diagnostic methods and a biomarker (i.e., TSPAN6), prognostic methods and a biomarker (i.e., TSPAN6), and therapy evaluators for Alzheimer's disease. In a specific embodiment, the biomarker of the disclosure is useful for detecting early-stage Alzheimer's disease. In addition, the disclosure provides compounds inhibiting the biological activity of TSPAN6, which can be used for the treatment of Alzheimer's disease. In a particular embodiment, a compound (or a molecule) inhibiting the biological activity of TSPAN6 is an antibody directed against TSPAN6. In yet another embodiment, a compound inhibiting the biological activity of TSPAN6 is an siRNA with a specificity for TSPAN6. In yet another embodiment, a compound inhibiting the biological activity of TSPAN6 is a peptide with a specificity for TSPAN6. In yet another embodiment, a compound inhibiting the biological activity of TSPAN6 is an extracellular fragment of TSPAN6 (e.g., the small or the large extracellular fragment of TSPAN6).

The nucleotide sequence of TSPAN6 is depicted in SEQ ID NO:1 and the amino acid sequence of TSPAN6 is depicted in SEQ ID NO:2.

Alternative names for tetraspanin 6 are tetraspanin TM4-D, tetraspanin TM4SF, T245 protein and putative NF-kappa-B-activating protein 321.

Without limiting the disclosure to a particular mechanism of action, it is believed that tetraspanin 6 influences the activity of gamma-secretase. Gamma-secretase is a high-molecular-weight complex containing Presenilin, Nicastrin, Aph-1 and Pen-2 that cleaves type I membrane proteins. These four components are necessary and sufficient for γ-secretase activity, but additional proteins might interact. According to topology predictions, tetraspanins have two extracellular domains (often referred to as the small extracellular loop and the large extracellular loop (LEL)) and three relatively short cytoplasmic regions. Previous experiments established that tetraspanins interact with one another and form a structural platform for the assembly of a novel class of microdomains (referred to as tetraspanin-enriched microdomains (TERM, TEM) or “tetraspanin webs”). It has been proposed that through a network of homotypic and heterotypic interactions, tetraspanins regulate the spatial juxtaposition of associated transmembrane receptors (e.g., integrins, receptor tyrosine kinases) on the plasma membrane, which results in coordination of signaling pathways. There is also emerging evidence that tetraspanins regulate biosynthetic maturation and trafficking of their associated partners.

In the disclosure, we have identified that when the activity of TSPAN6 is down-regulated in a neuronal cell, that the activity of the gamma-secretase is also down-regulated, as witnessed by the reduction of amyloid beta processing; the latter is reflected in a reduced production of Abeta40 and/or a reduced production of Abeta42. Thus, the wording “to reduce the biological activity of TSPAN6” is equivalent with the wording “the activity of TSPAN6 is down-regulated.” Accordingly, molecules that inhibit the expression of TSPAN6 can be used to manufacture a medicament for the treatment of Alzheimer's disease.

In a particular embodiment, the molecules that inhibit the expression of TSPAN6 are short interference RNA molecules. Thus, the disclosure provides the use of a short interference RNA (siRNA) hybridizing with an RNA molecule encoding a fragment of tetraspanin-6 (SEQ ID NO:1) for the manufacture of a medicament to prevent and/or to treat Alzheimer's disease.

In another embodiment, the disclosure provides a pharmaceutical composition comprising an effective amount of an isolated siRNA comprising a sense RNA strand and an antisense RNA strand, wherein the sense and the antisense RNA strands form an RNA duplex, and wherein the sense RNA strand comprises a nucleotide sequence identical to a target sequence of about 19 to about 25 contiguous nucleotides in SEQ ID NO:1. In particular, the disclosure, therefore, provides isolated siRNA comprising short double-stranded RNA from about 19 to about 25 nucleotides in length, that are targeted to the target mRNA of SEQ ID NO:1. The siRNA comprise a sense RNA strand and a complementary antisense RNA strand annealed together by standard Watson-Crick base-pairing interactions (hereinafter “base-paired”). The sense strand comprises a nucleic acid sequence that is identical to a target sequence contained within the target mRNA. The sense and antisense strands of the present siRNA can comprise two complementary, single-stranded RNA molecules or can comprise a single molecule in which two complementary portions are base-paired and are covalently linked by a single-stranded “hairpin” area. The term “isolated” means altered or removed from the natural state through human intervention. For example, an siRNA naturally present in a living animal is not “isolated,” but a synthetic siRNA, or an siRNA partially or completely separated from the coexisting materials of its natural state is “isolated.” An isolated siRNA can exist in substantially purified form, or can exist in a non-native environment such as, for example, a cell into which the siRNA has been delivered.

The siRNAs of the disclosure can comprise partially purified RNA, substantially pure RNA, synthetic RNA, or recombinantly produced RNA, as well as altered RNA that differs from naturally occurring RNA by the addition, deletion, substitution and/or alteration of one or more nucleotides. Such alterations can include addition of non-nucleotide material, such as to the end(s) of the siRNA or to one or more internal nucleotides of the siRNA, including modifications that make the siRNA resistant to nuclease digestion. One or both strands of the siRNA of the disclosure can also comprise a 3′ overhang. A “3′ overhang” refers to at least one unpaired nucleotide extending from the 3′-end of an RNA strand. Thus, in one embodiment, the siRNA of the disclosure comprises at least one 3′ overhang of from one to about six nucleotides (which includes ribonucleotides or deoxynucleotides) in length, preferably from one to about five nucleotides in length, more preferably from one to about four nucleotides in length, and particularly preferably from about one to about four nucleotides in length.

In the embodiment in which both strands of the siRNA molecule comprise a 3′ overhang, the length of the overhangs can be the same or different for each strand. In a most preferred embodiment, the 3′ overhang is present on both strands of the siRNA, and is two nucleotides in length. In order to enhance the stability of the present siRNAs, the 3′ overhangs can also be stabilized against degradation. In one embodiment, the overhangs are stabilized by including purine nucleotides, such as adenosine or guanosine nucleotides. Alternatively, substitution of pyrimidine nucleotides by modified analogues, e.g., substitution of uridine nucleotides in the 3′ overhangs with 2′-deoxythymidine, is tolerated and does not affect the efficiency of RNAi degradation. In particular, the absence of a 2′ hydroxyl in the 2′-deoxythymidine significantly enhances the nuclease resistance of the 3′ overhang in tissue culture medium.

The siRNAs of the disclosure can be targeted to any stretch of approximately 19-25 contiguous nucleotides in the target mRNA sequence (the “target sequence”), which sequence is depicted in SEQ ID NO:1. Techniques for selecting target sequences for siRNA are well known in the art. Thus, the sense strand of the present siRNA comprises a nucleotide sequence identical to any contiguous stretch of about 19 to about 25 nucleotides in the target mRNA. The siRNAs of the disclosure can be obtained using a number of techniques known to those of skill in the art. For example, the siRNAs can be chemically synthesized or recombinantly produced using methods known in the art. Preferably, the siRNA of the disclosure are chemically synthesized using appropriately protected ribonucleoside phosphoramidites and a conventional DNA/RNA synthesizer. The siRNA can be synthesized as two separate, complementary RNA molecules, or as a single RNA molecule with two complementary regions. Commercial suppliers of synthetic RNA molecules or synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA) and Cruachem (Glasgow, UK).

Alternatively, siRNA can also be expressed from recombinant circular or linear DNA plasmids using any suitable promoter. Suitable promoters for expressing siRNA of the disclosure from a plasmid include, for example, the U6 or H1 RNA pol III promoter sequences and the cytomegalovirus promoter. Selection of other suitable promoters is within the skill in the art. The recombinant plasmids of the disclosure can also comprise inducible or regulatable promoters for expression of the siRNA in a particular tissue or in a particular intracellular environment. The siRNA expressed from recombinant plasmids can either be isolated from cultured cell expression systems by standard techniques, or can be expressed intracellularly in neurons.

The siRNAs of the disclosure can also be expressed from recombinant viral vectors; e.g., intracellularly in neurons. The recombinant viral vectors comprise sequences encoding the siRNAs of the disclosure and any suitable promoter for expressing the siRNA sequences. Suitable promoters include, for example, the U6 or H1 RNA pol III promoter sequences and the cytomegalovirus promoter. Selection of other suitable promoters is within the skill in the art. The recombinant viral vectors of the disclosure can also comprise inducible or regulatable promoters for expression of the siRNA in the brain (e.g., in hippocampal neurons). As used herein, an “effective amount” of the siRNA is an amount sufficient to cause RNAi-mediated degradation of the target mRNA, or an amount sufficient to inhibit the progression of plaque formation (or amyloid-β 40/42 formation) in a subject. RNAi-mediated degradation of the target mRNA can be detected by measuring levels of the target mRNA or protein in the cells of a subject, using standard techniques for isolating and quantifying mRNA or protein as described above.

One skilled in the art can readily determine an effective amount of the siRNA of the disclosure to be administered to a given subject, by taking into account factors such as the size and weight of the subject; the extent of the disease penetration; the age, health and sex of the subject; the route of administration; and whether the administration is regional or systemic. Generally, an effective amount of the siRNA of the disclosure comprises an intracellular concentration of from about 1 nanomolar (nM) to about 100 nM, preferably from about 2 nM to about 50 nM, more preferably from about 2.5 nM to about 10 nM. It is contemplated that greater or lesser amounts of siRNA can be administered.

The methods can be used to prevent and/or to treat plaque formation of amyloid-β in the brain of patients suffering from Alzheimer's disease. For treating Alzheimer's disease, the siRNAs of the disclosure (one or more siRNAs directed to one, two or three targets) can be administered to a subject in combination with a pharmaceutical agent that is different from the present siRNA. Alternatively, the siRNA of the disclosure can be administered to a subject in combination with another therapeutic method designed to treat Alzheimer's disease. In the methods, the siRNAs (at least one or a combination of siRNAs directed against the target of the disclosure) can be administered to the subject either as naked siRNA, in conjunction with a delivery reagent, or as a recombinant plasmid or viral vector that expresses the siRNA.

In a particular embodiment, siRNAs are first bound to a peptide derived from Rabies virus that is coupled to a poly-Arginine stretch (YTIWMPENPRPGTPCDIFTNSRGKRASNGGGGRRRRRRRRR; SEQ ID NO:4) (see P. Kumar et al. (2007) Nature 448 (7149):39-43). Suitable delivery reagents for administration in conjunction with the present siRNA include the Minis Transit TKO lipophilic reagent; lipofectin; lipofectamine; cellfectin; or polycations (e.g., polylysine), or liposomes. A preferred delivery reagent is a liposome. Liposomes can increase the blood half-life of the siRNA. Liposomes suitable for use in the disclosure are formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. Preferably, the liposomes encapsulating the present siRNAs comprise a ligand molecule that can target the liposome to the brain. A preferred ligand is a peptide derived from Rabies Virus (YTIWMPENPRPGTPCDIFTNSRGKRASNG; SEQ ID NO:5) because this peptide ligand is capable of crossing the blood brain barrier and is also capable of crossing neuronal membranes. Particularly preferably, the liposomes encapsulating the present siRNA are modified so as to avoid clearance by the mononuclear macrophage and reticuloendothelial systems, for example, by having opsonization-inhibition moieties bound to the surface of the structure.

In one embodiment, a liposome of the disclosure can comprise both opsonization-inhibition moieties and a ligand. Opsonization-inhibiting moieties for use in preparing the liposomes of the disclosure are typically large hydrophilic polymers that are bound to the liposome membrane. As used herein, an opsonization-inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the macrophage-monocyte system (“MMS”) and reticuloendothelial system (“RES”). Liposomes modified with opsonization-inhibition moieties thus remain in the circulation much longer than unmodified liposomes. For this reason, such liposomes are sometimes called “stealth” liposomes. Preferably, the opsonization-inhibiting moiety is a PEG, PPG, or derivatives thereof. Liposomes modified with PEG or PEG-derivatives are sometimes called “PEGylated liposomes.”

The opsonization-inhibiting moiety can be bound to the liposome membrane by any one of numerous well-known techniques. For example, an N-hydroxysuccinimide ester of PEG can be bound to a phosphatidyl-ethanolamine lipid-soluble anchor, and then bound to a membrane. The siRNA can also be administered to a subject by gene gun, electroporation, or by other suitable parenteral or enteral administration routes. Suitable enteral administration routes include oral, rectal, or intranasal delivery. Suitable parenteral administration routes include intravascular administration (e.g., intravenous bolus injection, intravenous infusion, intra-arterial bolus injection, intra-arterial infusion and catheter instillation into the vasculature); peri- and intra-tissue injection (e.g., peri-tumoral and intra-tumoral injection, intra-retinal injection, or subretinal injection); subcutaneous injection or deposition including subcutaneous infusion (such as by osmotic pumps). In a particular embodiment, siRNAs are delivered through stereotactic injection into the brain (e.g., through intracerebroventricular injection).

The siRNAs of the disclosure can be administered in a single dose or in multiple doses. Where the administration of the siRNAs of the disclosure is by infusion, the infusion can be a single sustained dose or can be delivered by multiple infusions. One skilled in the art can also readily determine an appropriate dosage regimen for administering the siRNA (i.e., at least one siRNA) of the disclosure to a given subject. For example, the siRNA can be administered to the subject once, for example, as a single injection or deposition directly into the brain. Alternatively, the siRNA can be administered once or twice daily to a subject for a period of from about three to about twenty-eight days, more preferably from about seven to about ten days. Where a dosage regimen comprises multiple administrations, it is understood that the effective amount of siRNA administered to the subject can comprise the total amount of siRNA administered over the entire dosage regimen.

The siRNAs of the disclosure are preferably formulated as pharmaceutical compositions prior to administering to a subject, according to techniques known in the art. Pharmaceutical compositions of the invention are characterized as being at least sterile and pyrogen-free. As used herein, “pharmaceutical formulations” include formulations for human and veterinary use. Methods for preparing pharmaceutical compositions of the disclosure are within the skill in the art, for example, as described in Remington's Pharmaceutical Science, 17th ed., Mack Publishing Company, Easton, Pa. (1985), the entire disclosure of which is herein incorporated by reference. The pharmaceutical formulations comprise at least one siRNA of the disclosure (e.g., 0.1 to 90% by weight), or a physiologically acceptable salt thereof, mixed with a physiologically acceptable carrier medium. Preferred physiologically acceptable carrier media are water, buffered water, normal saline, 0.4% saline, 0.3% glycine, hyaluronic acid and the like.

Pharmaceutical compositions of the disclosure can also comprise conventional pharmaceutical excipients and/or additives. Suitable pharmaceutical excipients include stabilizers, antioxidants, osmolality-adjusting agents, buffers, and pH-adjusting agents. Suitable additives include physiologically biocompatible buffers (e.g., tromethamine hydrochloride), additions of chelants (such as, for example, DTPA or DTPA-bisamide) or calcium chelate complexes (as, for example, calcium DTPA, CaNaDTPA-bisamide), or, optionally, additions of calcium or sodium salts (for example, calcium chloride, calcium ascorbate, calcium gluconate or calcium lactate). Pharmaceutical compositions of the disclosure can be packaged for use in liquid form or can be lyophilized. For solid compositions, conventional nontoxic solid carriers can be used; for example, pharmaceutical grades of mannitol, lactose, starch, magnesium stearate, sodium saccharin, talcum, cellulose, glucose, sucrose, magnesium carbonate, and the like. For example, a solid pharmaceutical composition for oral administration can comprise any of the carriers and excipients listed above and 10-95%, preferably 25%-75%, of one or more siRNAs of the disclosure. A pharmaceutical composition for aerosol (inhalational) administration can comprise 0.01-20% by weight, preferably 1%-10% by weight, of one or more siRNAs of the disclosure encapsulated in a liposome as described above. A carrier can also be included as desired; e.g., lecithin for intranasal delivery.

In yet another specific embodiment, the disclosure uses an antibody binding to tetraspanin-6 (SEQ ID NO:2) for the manufacture of a medicament to prevent and/or to treat Alzheimer's disease.

In yet another specific embodiment of the disclosure, the antibody specifically binds to the one of the two extracellular domains of TSPAN6. Without limiting the disclosure to a particular mechanism, it is believed that an antibody binding to the extracellular domain of TSPAN6 will prevent the interaction between TSPAN6 and gamma-secretase. It is also believed that an antibody binding the large extracellular domain of TSPAN6 (i.e., EC2) will prevent the dimerization of TSPAN6 and thereby reducing the biological activity of TSPAN6.

The terms “antibody” or “antibodies” relate to an antibody characterized as being specifically directed against SEQ ID NO:2 or any functional derivative thereof, with the antibodies being preferably monoclonal antibodies, or an antigen-binding fragment thereof, of the F(ab′)₂, F(ab) or single chain Fv type, or any type of recombinant antibody derived thereof. These antibodies of the disclosure, including specific polyclonal antisera prepared against SEQ ID NO:2 or any functional derivative thereof, have no cross-reactivity to other proteins.

The monoclonal antibodies of the disclosure can, for instance, be produced by any hybridoma liable to be formed according to classical methods from splenic cells of an animal, particularly of a mouse or rat immunized against SEQ ID NO:2 or any functional derivative thereof, and of cells of a myeloma cell line, and to be selected by the ability of the hybridoma to produce the monoclonal antibodies recognizing SEQ ID NO:2, or any functional derivative thereof, which have been initially used for the immunization of the animals. The monoclonal antibodies according to this embodiment of the disclosure may be humanized versions of the mouse monoclonal antibodies made by means of recombinant DNA technology, departing from the mouse and/or human genomic DNA sequences coding for H and L chains or from cDNA clones coding for H and L chains.

Alternatively, the monoclonal antibodies according to this embodiment of the disclosure may be human monoclonal antibodies. Such human monoclonal antibodies are prepared, for instance, by means of human peripheral blood lymphocytes (PBL) repopulation of severe combined immune deficiency (SCID) mice as described in PCT/EP 99/03605 or by using transgenic non-human animals capable of producing human antibodies as described in U.S. Pat. No. 5,545,806. Also, fragments derived from these monoclonal antibodies, such as Fab, F(ab)′₂ and scFv (“single chain variable fragment”), providing they have retained the original binding properties, form part of the disclosure. Such fragments are commonly generated by, for instance, enzymatic digestion of the antibodies with papain, pepsin, or other proteases. It is well known to the person skilled in the art that monoclonal antibodies, or fragments thereof, can be modified for various uses. The antibodies involved in the disclosure can be labeled by an appropriate label of the enzymatic, fluorescent, or radioactive type. In a particular embodiment, the antibodies against SEQ ID NO:2 or a functional fragment thereof are derived from camels. Camel antibodies are fully described in WO94/25591, WO94/04678 and in WO97/49805.

In yet another particular embodiment, the disclosure contemplates an extracellular fragment of TSPAN6 for the treatment of AD. Examples of such fragments are the small extracellular domain of TSPAN6 (EC1) and the large extracellular domain of TSPAN6 (EC2). In a specific embodiment, the disclosure provides the large extracellular fragment of TSPAN6 or an amino acid sequence derived from the large extracellular fragment of at least 15 amino acids. The large extracellular fragment of TSPAN6 is depicted in SEQ ID NO:3.

In a particular embodiment, the disclosure also contemplates non-antibody binding proteins against TSPAN6, in particular, binding to the extracellular domains of TSPAN6. These “non-antibody binding proteins” refer to compounds (often designated as antibody mimics) that use non-immunoglobulin protein scaffolds, including adnectins, avimers, aptamers, single chain polypeptide binding molecules, and antibody-like binding peptidomimetics. These other compounds have been developed that target and bind to targets in a manner similar to antibodies. Certain of these “antibody mimics” use non-immunoglobulin protein scaffolds as alternative protein frameworks for the variable regions of antibodies. Non-limiting examples are described in U.S. Pat. No. 5,260,203, U.S. Pat. No. 6,818,418, U.S. Pat. No. 7,115,396 and U.S. Pat. No. 5,770,380.

The term “medicament to treat” relates to a composition comprising molecules as described above and a pharmaceutically acceptable carrier or excipient (both terms can be used interchangeably) to prevent and/or to treat Alzheimer's disease. Suitable carriers or excipients known to the skilled man are saline, Ringer's solution, dextrose solution, Hank's solution, fixed oils, ethyl oleate, 5% dextrose in saline, substances that enhance isotonicity and chemical stability, buffers and preservatives. Other suitable carriers include any carrier that does not itself induce the production of antibodies harmful to the individual receiving the composition such as proteins, polysaccharides, polylactic acids, polyglycolic acids, polymeric amino acids and amino acid copolymers.

The “medicament” may be administered by any suitable method within the knowledge of the skilled man. One route of administration is parenterally. In parental administration, the medicament of this disclosure will be formulated in a unit dosage injectable form such as a solution, suspension or emulsion, in association with the pharmaceutically acceptable excipients as defined above. However, the dosage and mode of administration will depend on the individual. Generally, the medicament is administered so that the antibody of the disclosure is given at a dose between 1 μg/kg and 10 mg/kg, more preferably between 10 μg/kg and 5 mg/kg, most preferably between 0.1 and 2 mg/kg. Preferably, it is given as a bolus dose. Continuous infusion may also be used. If so, the medicament may be infused at a dose between 5 and 20 μg/kg/minute, more preferably between 7 and 15 μg/kg/minute.

It is clear to the person skilled in the art that the use of a therapeutic composition comprising, for example, an antibody against SEQ ID NO:2 for the manufacture of a medicament to prevent and/or to treat Alzheimer's disease can be administered by any suitable means, including, but not limited to, parenteral, subcutaneous, intraperitoneal, intrapulmonary, intracerebroventricular and intranasal administration. Parenteral infusions include intramuscular, intravenous, intra-arterial, intraperitoneal, or subcutaneous administration. In addition, the therapeutic composition is suitably administered by pulse infusion, particularly with declining doses of the antibody.

### Diagnostic Applications of the Disclosure

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or +10%, more preferably ±5%, even more preferably ±1%, and still more preferably +0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics that are normal or expected for one cell or tissue type, might be abnormal for a different cell or tissue type.

As used herein, an “immunoassay” refers to any binding assay that uses an antibody capable of binding specifically to a target molecule to detect and quantify the target molecule.

By the term “specifically binds,” as used herein with respect to an antibody, is meant an antibody that recognizes a specific antigen (e.g., TSPAN6), but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross-reactivity does not itself alter the classification of an antibody as specific. In some instances, the terms “specific binding” or “specifically binding” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody.

As used herein, “biomarker” in the context of the disclosure encompasses, without limitation, proteins, nucleic acids, and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. Biomarkers also encompass non-blood borne factors or non-analyte physiological biomarkers of health status, such as clinical parameters, as well as traditional laboratory risk factors. Biomarkers also include any calculated indices created mathematically or combinations of any one or more of the foregoing measurements, including temporal trends and differences.

As used herein, the teen “data” in relation to one or more biomarkers, or the term “biomarker data” generally refers to data reflective of the absolute and/or relative abundance (level) of a product of a biomarker in a sample.

As used herein, the term “dataset” in relation to one or more biomarkers refers to a set of data representing levels of each of one or more biomarker products of a panel of biomarkers in a reference population of subjects. A dataset can be used to generate a formula/classifier of the disclosure. According to one embodiment, the dataset need not comprise data for each biomarker product of the panel for each individual of the reference population. For example, the “dataset” when used in the context of a dataset to be applied to a formula can refer to data representing levels of products of each biomarker for each individual in one or more reference populations, but as would be understood can also refer to data representing levels of products of each biomarker for 99%, 95%, 90%, 85%, 80%, 75%, 70% or less of the individuals in each of the one or more reference populations and can still be useful for purposes of applying to a formula.

“Differentially increased expression” or “up-regulation” refers to biomarker product levels that are at least 10% or more, for example, 20%, 30%, 40%, or 50%, 60%, 70%, 80%, 90% higher or more, and/or 1.1-fold, 1.2-fold, 1.4-fold, 1.6-fold, 1.8-fold higher or more, than a control. As used herein, an “immunoassay” refers to any binding assay that uses an antibody capable of binding specifically to a target molecule to detect and quantify the target molecule. By the term “specifically binds,” as used herein with respect to an antibody, is meant an antibody that recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross-reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A,” the presence of a molecule containing epitope A (or free, unlabeled A), in a reaction containing labeled “A” and the antibody, will reduce the amount of labeled A bound to the antibody. As used herein, “biomarker” in the context of the disclosure encompasses, without limitation, proteins, nucleic acids, and metabolites, together with their polymorphisms, mutations, variants, modifications, subunits, fragments, protein-ligand complexes, and degradation products, elements, related metabolites, and other analytes or sample-derived measures. Biomarkers can also include mutated proteins or mutated nucleic acids. Biomarkers also encompass non-blood-borne factors or non-analyte physiological markers of health status, such as clinical parameters, as well as traditional laboratory risk factors. Biomarkers also include any calculated indices created mathematically or combinations of any one or more of the foregoing measurements, including temporal trends and differences.

In the disclosure, it is shown that the expression of the TSPAN6 gene is specifically elevated in the cerebral prefrontal cortex of Alzheimer's disease patients. In addition, a positive correlation was identified between the mRNA levels of TSPAN6 and the Braak stages of the disease. Therefore, the gene identified herein as well as its transcription and translation products have diagnostic utility as a biomarker for Alzheimer's disease by measuring the expression level of TSPAN6 between a subject-derived sample and a control sample that is derived from a subject not suffering from Alzheimer's disease. In certain embodiments, the method comprises the step of obtaining a sample from a subject suspected of having AD and assessing the level of TSPAN6 in the sample. Thus, the disclosure relates to a biomarker of Alzheimer's disease, methods for diagnosis of Alzheimer's Disease, methods of deter mining predisposition to Alzheimer's Disease, methods of monitoring progression/regression of Alzheimer's Disease, methods of assessing efficacy of compositions for treating Alzheimer's Disease, methods of screening compositions for activity in modulating biomarkers of Alzheimer's Disease, as well as other diagnostic methods based on the biomarker of Alzheimer's Disease.

The “Braak stages” of the “Braak six-part staging system” for neuropathologists focuses on the time and space issues of the sequence of progression of injured neurons bearing neurofibrillary tangles in Alzheimer's autopsy brain tissues. Autopsy brain studies demonstrate that based on the single parameter of tangles, autopsy brains with tangles confined to small regions of the entorhinal cortex (proximate to the hippocampus) comprise Braak Stage 1. Stage 1 patients are never demented. Brains with widespread “tangle bearing” neurons in the higher neocortex and occipital cortex regions are Stage 6. Stage 6 patients are always demented. Stages 2-5 in the Braak system are intermediary points in the journey from intact brain function to total incapacitation.

In certain embodiments, the disclosure further provides methods for permitting refinement of disease diagnosis, disease risk prediction, and clinical management of individuals associated with a neurodegenerative disorder. In a particular embodiment, the biomarker can be used to detect AD in a population of subjects suffering from dementia. In yet another embodiment, the biomarker can be used to detect AD in a population of subjects suffering from other neurodegenerative disorders (e.g., frontotemporal lobe dementia and other types of dementia).

In a specific embodiment, age, gender, and ApoE genotype (e2/e2, e2/e3, e2/e4, e3/e3, e3/e4 and e4/e4) are additional factors that are considered in identifying an individual for Alzheimer's disease.

In a particular embodiment, an immunoassay is used for the assessment of a biomarker level. In another embodiment, a luminex technology multiplex immunoassay is used to assess the biomarker level.

In a specific embodiment, a method is provided for detecting or diagnosing the presence of Alzheimer's disease or a predisposition to Alzheimer's disease in a subject comprising determining the expression level of TSPAN6 in a biological sample derived from the subject, wherein an increase of the level compared to a normal control of the gene indicates that the subject suffers from or is at risk of developing Alzheimer's disease, wherein the expression level is determined by any one method selected from the group consisting of: a) detecting a mRNA of TSPAN6, b) detecting a protein encoded by TSPAN6 and c) detecting the biological activity of the protein encoded by TSPAN6.

In another embodiment, a method of diagnosing Alzheimer's disease in an individual comprises the steps of obtaining a first biological sample from the individual at a first time; assessing the level of TSPAN6 in the biological sample to obtain a baseline level; obtaining a second biological sample from the individual at a second time and assessing the level of TSPAN6 in the second biological sample to obtain a second level. If the second level of TSPAN6 is significantly enhanced compared to the baseline level, the individual is at an increased risk of developing or having Alzheimer's disease. In one embodiment, the second level is also compared to a reference population of individuals without Alzheimer's disease. If the second level is significantly altered compared to the level derived from a reference population, the individual is at an increased risk of developing or having Alzheimer's disease.

In still further embodiments, the disclosure provides methods of monitoring the TSPAN6 level in a biological sample to evaluate the progress of a therapeutic treatment of Alzheimer's disease.

In another embodiment, the disclosure provides methods for selecting a patient that is most likely to respond to treatment.

The “biological sample” or “sample derived from a subject” means a biological material isolated from an individual. The biological sample may contain any biological material suitable for detecting TSPAN6, and may comprise cellular and/or non-cellular material obtained from the individual. Accordingly, the biological samples include, but are not limited to, bodily tissues and fluids, for example, blood, serum, plasma, sputum, urine, cerebrospinal fluid (CSF), saliva, pleural effusion, nipple aspiration fluid, tears, etc.

The disclosure also provides methods for screening an individual to determine if the individual is at increased risk of having Alzheimer's disease. Individuals found to be at increased risk can be given appropriate therapy and monitored using the methods of the disclosure. Other methods and kits useful in practicing the methods of the disclosure are provided herein.

According to the disclosure, the expression level of TSPAN6 in the subject-derived biological sample is determined. The expression level can be determined at the transcription (nucleic acid) product level, using methods known in the art. For example, the mRNA of TSPAN6 gene can be quantified using probes by hybridization methods (e.g., Northern blot analysis). The detection can be carried out on a chip or an array. The use of an array can be for detecting the expression level of a plurality of genes (e.g., various neurological disease-specific genes) including the TSPAN6 gene. Those skilled in the art can prepare such probes utilizing the sequence information of the TSPAN6 (SEQ ID NO:1). For example, the cDNA of the TSPAN6 gene can be used as a probe. If necessary, the probe can be labeled with a suitable label, for example, dyes, fluorescent and isotopes, and the expression level of the gene can be detected as the intensity of the hybridized labels. Furthermore, the transcription product of the TSPAN6 gene can be quantified using primers by amplification-based detection methods (e.g., RT-PCR). Such primers can also be prepared based on the available sequence information of the gene. Specifically, a probe or primer used for the method hybridizes under stringent, moderately stringent, or low stringent conditions to the mRNA of the TSPAN6 gene.

Alternatively, the translation product (i.e., the protein) of the TSPAN6 gene can be detected for the diagnosis of the disclosure. For example, the quantity of the TSPAN6 protein can be determined. There are numerous known methods and kits for measuring the amount or concentration of a protein in a sample, including as non-limiting examples, ELISA, Western blot, absorption measurement, colorimetric determination, Lowry assay, Bicinchoninic acid assay, or a Bradford assay. Commercial kits include PROTEOQWEST™ Colorimetric Western Blotting Kits (Sigma-Aldrich, Co.), QUANTIPRO™ bicinchoninic acid (BCA) Protein Assay Kit (Sigma-Aldrich, Co.), FLUOROPROFILE™ Protein Quantification Kit (Sigma-Aldrich, Co.), the Coomassie Plus—The Better Bradford Assay (Pierce Biotechnology, Inc.), and the Modified Lowry Protein Assay Kit (Pierce Biotechnology, Inc.). In certain embodiments, the protein concentration is measured using a luminex-based multiplex immunoassay panel. However, the disclosure should not be limited to any particular assay for assessing the level of the biomarker of the disclosure. That is, any currently known assay used to detect protein levels can be used to detect the biomarkers of the disclosure. Methods of quantitatively assessing the level of a protein in a biological sample such as CSF, urine or saliva are well known in the art. In some embodiments, assessing the level of a protein involves the use of a detector molecule for the biomarker. Detector molecules can be obtained from commercial vendors or can be prepared using conventional methods available in the art. Exemplary detector molecules include, but are not limited to, an antibody that binds specifically to the biomarker, a naturally occurring cognate receptor, or functional domain thereof, for the biomarker, or a small molecule that binds specifically to the biomarker.

In a preferred embodiment, the level of a biomarker is assessed using an antibody. Thus, non-limiting exemplary methods for assessing the level of a biomarker in a biological sample include various immunoassays, for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See, e.g., Harlow et al., 1988, Antibodies: A Laboratory Manual, Cold Spring Harbor, N.Y.; Harlow et al., 1999, Using Antibodies: A Laboratory Manual, Cold Spring Harbor Laboratory Press, NY.

The generation of polyclonal antibodies is accomplished by inoculating the desired animal with an antigen and isolating antibodies that specifically bind the antigen therefrom. Monoclonal antibodies directed against the biomarkers identified herein may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). For use in preparing an antibody, a biomarker may be purified from a biological source that endogenously comprises the biomarker, or from a biological source recombinantly engineered to produce or over-produce the biomarker, using conventional methods known in the art. Preferably, antibodies are generated against the human homologue of TSPAN6. Nucleic acid encoding the monoclonal antibody obtained using the procedures described herein may be cloned and sequenced using technology that is available in the art, and is described, for example, in Wright et al. (1992, Critical Rev. Immunol. 12(3,4):125-168) and the references cited therein. Further, the antibody useful in the practice of the disclosure may be “humanized.”

Other methods for assessing the level of a protein include chromatography (e.g., HPLC, gas chromatography, liquid chromatography) and mass spectrometry (e.g., MS, MS-MS). For instance, a chromatography medium comprising a cognate receptor for the biomarker or a small molecule that binds to the biomarker can be used to substantially isolate the biomarker from the biological sample. Small molecules that bind specifically to a biomarker can be identified using conventional methods in the art, for instance, screening of compounds using combinatorial library methods known in the art, including biological libraries, spatially addressable parallel solid phase or solution phase libraries, synthetic library methods requiring deconvolution, the “one-bead one-compound” library method, and synthetic library methods using affinity chromatography selection. The level of substantially isolated protein can be quantitated directly or indirectly using a conventional technique in the art such as spectrometry, Bradford protein assay, Lowry protein assay, biuret protein assay, or bicinchoninic acid protein assay, as well as immunodetection methods.

In a particular embodiment, the diagnostic application of the disclosure differentiates the presence of Alzheimer's disease in a patient's sample from the presence of other neurodegenerative diseases such as, for example, Lewy Body Dementia (LBD) and frontotemporal lobe dementia (FTLD).

### Determination of the Status of Alzheimer's Disease

The disclosure is based on the detection or a quantification of the biomarker of the disclosure or is based on a biomarker profile (consisting of the biomarker of the disclosure) or signature (consisting of the biomarker of the disclosure) determined for biological samples from individuals diagnosed with Alzheimer's Disease as well as from one or more other groups of control individuals (e.g., healthy control subjects not diagnosed with Alzheimer's Disease; or alternatively, other patients suffering from dementia; or other patients suffering from other neurodegenerative diseases). The profile for Alzheimer's Disease is compared to the profile for biological samples from the one or more other groups of control individuals. The biomarker differentially present, at a level that is statistically significant, in the profile of Alzheimer's Disease samples as compared to another group (e.g., healthy control subjects not diagnosed with Alzheimer's Disease) is identified as a biomarker to distinguish those groups.

The reference level used for comparison with the measured level for the AD biomarker may vary, depending on one aspect of the disclosure being practiced, as will be understood from the foregoing discussion. For detection of AD, the “reference level” is typically a predetermined reference level, such as an average of levels obtained from a population that is not afflicted with AD, but in some instances, the reference level can be a mean or median level from a group of individuals including AD patients. In some instances, the predetermined reference level is derived from (e.g., is the mean or median of) levels obtained from an age-matched population. In some instances, the age-matched population comprises individuals with non-AD neurodegenerative disorders. In some instances, the reference level may be a historical reference level for the particular patient (e.g., the biomarker level that was obtained from a sample derived from the same individual, but at an earlier point in time). In some instances, the predetermined reference level is derived from (e.g., is the mean or median of) levels obtained from an age-matched population. Age-matched populations (from which reference values may be obtained) are ideally the same age as the individual being tested, but approximately age-matched populations are also acceptable. Approximately age-matched populations may be within 1, 2, 3, 4, or 5 years of the age of the individual tested, or may be groups of different ages that encompass the age of the individual being tested. Approximately age-matched populations may be in 2, 3, 4, 5, 6, 7, 8, 9, or 10-year increments (e.g., a “5-year-increment” group, which serves as the source for reference values for a 62-year-old individual might include 58- to 62-year-old individuals, 59- to 63-year-old individuals, 60- to 64-year-old individuals, 61- to 65-year-old individuals, or 62- to 66-year-old individuals.

The level(s) of the biomarker may be compared to Alzheimer's Disease-positive and/or Alzheimer's Disease-negative reference levels using various techniques, including a simple comparison (e.g., a manual comparison) of the level of the biomarker in the biological sample to Alzheimer's Disease-positive and/or Alzheimer's Disease-negative reference levels. The level of the biomarker in the biological sample may also be compared to Alzheimer's Disease-positive and/or Alzheimer's Disease-negative reference levels using one or more statistical analyses. Statistical models useful in the disclosure include, but are not limited to, Logistic Regression, Boosted Tree Models, Flexible Discriminant Analysis (FDA), K-Nearest Neighbors (KNN), Naïve Bayes, Partial Least Squares (PLS), Random Forests, Shrunken Centroids, Sparse Partial Least Squares and Support Vector Machines approaches.

In specific embodiments, age, gender, and ApoE genotype (e2/e2, e2/e3, e2/e4, e3/e3, e3/e4 and e4/e4) are additional factors that are considered in identifying an individual for Alzheimer's disease.

In other specific embodiments, the biomarker of the disclosure can be combined with additional confirmatory CSF and imaging testing.

The biomarker of the disclosure can be used in diagnostic tests to assess the status of Alzheimer's disease in an individual, e.g., to diagnose Alzheimer's disease or to assess the degree of Alzheimer's disease in the individual. The phrase “Alzheimer's disease status” includes any distinguishable manifestation of the disease, including non-Alzheimer's disease, e.g., normal or non-demented. For example, disease status includes, without limitation, the presence or absence of Alzheimer's disease (e.g., Alzheimer's disease v. non-Alzheimer's disease), the risk of developing disease, the stage of the disease, the progress of disease (e.g., progress of disease or remission of disease over time) and the effectiveness or response to treatment of disease. Based on this status, further procedures may be indicated, including additional diagnostic tests or therapeutic procedures or regimens.

The ability of a diagnostic test to correctly predict the status is commonly measured based on the sensitivity of the assay, the specificity of the assay or the area under a receiver-operated characteristic (“ROC”) curve. Sensitivity is the percentage of true positives that are predicted by a test to be positive, while specificity is the percentage of true negatives that are predicted by a test to be negative. An ROC curve provides the sensitivity of a test. The greater the area under the ROC curve, the more powerful the predictive value of the test. Other useful measures of the utility of a test are positive predictive value and negative predictive value. Positive predictive value is the percentage of people who test positive that is actually positive. Negative predictive value is the percentage of people who test negative that is actually negative.

As apparent from the example disclosed herein, diagnostic tests that use the biomarker of the disclosure exhibit a sensitivity and specificity of at least 75%, at least 80%, at least 85%, at least 90%, at least 95%, at least 98% and about 100%. In some instances, screening tools of the disclosure exhibit a high sensitivity of at least 75%, at least 80%, at least 85%, at least 90%, at least 95%, at least 98% and about 100%. Without wishing to be bound by any particular theory, it is believed that screening tools should exhibit high sensitivity, but specificity can be low. However, diagnostics should have high sensitivity and specificity.

While an individual biomarker is a useful diagnostic biomarker, it is well known that a combination of biomarkers can provide greater predictive value of a particular status than a single biomarker alone. Specifically, the detection of a plurality of biomarkers in a sample can increase the sensitivity and/or specificity of the test. A combination of at least two biomarkers is sometimes referred to as a “biomarker profile” or “biomarker fingerprint.” In addition, the methods disclosed herein using the biomarker may be used in combination with clinical diagnostic measures of Alzheimer's Disease and/or other neurodegenerative diseases. Combinations with clinical diagnostics may facilitate the disclosed methods, or confirm results of the disclosed methods (for example, facilitating or confirming diagnosis, monitoring progression or regression, and/or determining predisposition to Alzheimer's Disease). Determining Alzheimer's disease status typically involves classifying an individual into one of two or more groups based on the results of the diagnostic test. The diagnostic tests described herein can be used to classify an individual into a number of different states. In one embodiment, the disclosure provides methods for determining the presence or absence of Alzheimer's disease in an individual (status: Alzheimer's disease v. non-Alzheimer's disease). The presence or absence of Alzheimer's disease is determined by measuring at least the relevant biomarker in samples obtained from individuals and then either submitting them to a classification algorithm or comparing them with a reference amount and/or pattern of at least one biomarker that is associated with the particular risk level.

In another embodiment, the disclosure provides methods for determining the risk of developing disease in an individual. Biomarker amounts or patterns are characteristic of various risk states, e.g., high, medium or low. The risk of developing Alzheimer's disease is determined by measuring at least the relevant biomarker in a sample obtained from individuals and then either submitting them to a classification algorithm or comparing them with a reference amount and/or pattern of biomarkers that is associated with the particular risk level.

In yet another embodiment, the disclosure provides methods for determining the stage of Alzheimer's disease in an individual. Each stage of the disease can be characterized by the amount of the biomarker of the disclosure or relative amounts of a set of biomarkers (i.e., a pattern) that are found in a sample obtained from the individual. The stage of Alzheimer's disease is determined by measuring the relevant biomarker or biomarkers and then either submitting them to a classification algorithm or comparing them with a reference amount and/or pattern of biomarkers that is associated with the particular stage.

In another embodiment, the disclosure provides methods for determining the course of Alzheimer's disease in an individual. Disease course refers to changes in disease status over time, including disease progression (worsening) and disease regression (improvement). Over time, the amounts or relative amounts (e.g., the pattern) of the biomarkers change. For example, levels of various biomarkers of the disclosure increase with progression of disease. Accordingly, this method involves measuring the level of one or more biomarkers in an individual at two or more different time points, e.g., a first time and a second time, and comparing the change in amounts. The course of disease is determined based on these comparisons.

In a specific embodiment, the levels of biomarker of the disclosure increase with disease progression. In this method, the level of the biomarker in a sample from an individual is measured at two or more different time points, e.g., a first time and a second time, and the change in levels, if any is assessed. The course of disease is determined based on these comparisons. Similarly, changes in the rate of disease progression (or regression) may be monitored by measuring the level of one or more biomarkers at different times and calculating the rate of change in biomarker levels. The ability to measure disease state or rate of disease progression is important for drug treatment studies where the goal is to slow down or arrest disease progression using therapy. Additional embodiments of the disclosure relate to the communication of the results or diagnoses or both to technicians, physicians or patients, for example. In certain embodiments, computers are used to communicate results or diagnoses or both to interested parties, e.g., physicians and their patients.

In certain embodiments, the methods of the disclosure further comprise managing individual treatment based on their disease status. Such management includes the actions of the physician or clinician subsequent to determining Alzheimer's disease status. For example, if a physician makes a diagnosis of Alzheimer's disease, then a certain regimen of treatment, such as prescription or administration of the therapeutic drug might follow. Alternatively, a diagnosis of non-Alzheimer's disease might be followed by further testing to determine any other diseases that the patient might be suffering from. Also, if the test is inconclusive with respect to Alzheimer's disease status, further tests may be called for.

In a preferred embodiment of the disclosure, a diagnosis based on the presence or absence or relative levels in the biological sample of an individual of the relevant biomarker disclosed herein is communicated to the individual as soon as possible after the diagnosis is obtained.

According to yet another aspect, the disclosure provides a method of assessing efficacy of a treatment of Alzheimer's disease in a patient comprising: a) determining a baseline level of the at least one biomarker in a first sample obtained from the patient before receiving the treatment; b) determining the level of the at least one biomarker in a second sample obtained from the patient after receiving the treatment; wherein an alteration in the levels of the at least one biomarker in the post-treatment sample is correlated with a positive treatment outcome.

### Assays for the Diagnosis of Alzheimer's Disease

The experiments disclosed herein are designed to develop an assay to identify the biomarker of the disclosure for diagnosing, screening, monitoring and staging neurodegenerative diseases such as Alzheimer's disease that are fast, more accurate, and less expensive. The disclosure contemplates that a diagnostic assay can be developed that can detect, among others, early onset of Alzheimer's disease. Detection of early onset of Alzheimer's disease is believed to increase the success rate of the individual being successfully treated for Alzheimer's disease. The diagnostic method of the disclosure can be applied to subjects who have been previously diagnosed with Alzheimer's disease, those who are suspected of having Alzheimer's disease, and those at risk of developing Alzheimer's disease. For example, patients diagnosed with dementia, in particular, those patients who were previously clinically normal, are suitable subjects. However, it is not intended that the disclosure be limited to use with any particular subject types.

According to some embodiments, the subject is a human subject.

According to certain embodiments, the subject is selected from the group consisting of subjects displaying pathology resulting from Alzheimer's disease, subjects suspected of displaying pathology resulting from Alzheimer's disease, and subjects at risk of displaying pathology resulting from Alzheimer's disease.

According to another embodiment, the Alzheimer's disease diagnosed using the method of the disclosure is selected from the group consisting of late onset Alzheimer's disease, early onset Alzheimer's disease, familial Alzheimer's disease and sporadic Alzheimer's disease.

Early-onset Alzheimer's disease (EOAD) is a rare form of Alzheimer's disease in which individuals are diagnosed with the disease before age 65. Less than 10% of all Alzheimer's disease patients have EOAD. Younger individuals who develop Alzheimer's disease exhibit more of the brain abnormalities that are normally associated with Alzheimer's disease. EOAD is usually familial and follows an autosomal dominant inheritance pattern. To date, mutations in several genes including amyloid precursor protein (APP) on chromosome 21, presenilin 1 (PSEN1) on chromosome 14 and presenilin 2 (PSEN2) on chromosome 1 have been identified in families with EOAD. Most of the pathogenic mutations in the APP and presenilin genes are associated with abnormal processing of APP, which leads to the overproduction of toxic Aβ42.

Late-onset Alzheimer's disease (LOAD) is the most common form of Alzheimer's disease, accounting for about 90% of cases and usually occurring after age 65. LOAD strikes almost half of all individuals over the age of 85 and may or may not be hereditary. It is a complex and multifactorial disease with the possible involvement of several genes.

Based on the disclosure presented herein, a skilled artisan would understand that a profile of the biomarker of the disclosure, optionally in combination with other suitable biomarkers described in the art for Alzheimer's disease, can be detected in a suitable sample and the profile identified in the sample can differentiate AD from healthy controls and other forms of dementia. The profiles for Alzheimer's disease includes the biomarker disclosed herein. In some instances, the profile for Alzheimer's disease is a combination of biomarkers and other factors of Alzheimer's disease disclosed herein. For example, the biomarker of the disclosure, in combination with other factors such as age, gender, ApoE genotype (e2/e2, e2/e3, e2/e4, e3/e3, e3/e4 and e4/e4), can improve diagnostic and screening accuracy. The biomarker can also be combined with cognitive tests such as a simple memory test to improve diagnostic and screening accuracy. In some instances, the biomarker of the disclosure can be combined with additional confirmatory CSF and imaging testing.

For example, the biomarker of the disclosure can be combined with existing criteria for dementia to improve diagnostic and screening accuracy of Alzheimer's disease. Dementia is the decline of memory and other cognitive functions in comparison with the patient's previous level of function as determined by a history of decline in performance and by abnormalities noted from clinical examination and neuropsychological tests. A diagnosis of dementia cannot be made when consciousness is impaired by delirium, drowsiness, stupor, or coma or when other clinical abnormalities prevent adequate evaluation of mental status. Dementia is a diagnosis based on behavior and cannot be determined by computerized tomography, electroencephalography, or other laboratory instructions, although specific causes of dementia may be identified by these means.

In some instances, the biomarker of the disclosure can be combined with existing criteria for Alzheimer's disease. A clinical diagnosis of probable Alzheimer's disease can be made with confidence if there is a typical insidious onset of dementia with progression and if there are no other systemic or brain diseases that could account for the progressive memory and other cognitive deficits. Among the disorders that must be excluded are manic depressive disorder, Parkinson's disease, multi-infarct dementia, and drug intoxication; less commonly encountered disorders that may cause dementia include thyroid disease, pernicious anemia, luetic brain disease and other chronic infections of the nervous system, subdural hematoma, occult hydrocephalus, Huntington's disease, Creutzfeldt-Jakob disease, and brain tumors.

A diagnosis of definite Alzheimer's disease requires histopathologic confirmation. A clinical diagnosis of possible Alzheimer's disease may be made in the presence of other significant diseases, particularly if, on clinical judgment, Alzheimer's disease is considered the more likely cause of the progressive dementia. The clinical diagnosis of possible rather than probable Alzheimer's disease may be used if the presentation or course is somewhat aberrant. The information needed to apply these criteria is obtained by standard methods of examination: the medical history; neurologic; psychiatric, and clinical examinations; neuropsychological tests; and laboratory studies.

### Kits for the Diagnosis of Alzheimer's Disease

In a particular embodiment, a kit is envisaged for every method disclosed in the application. The following description of a kit useful for diagnosing Alzheimer's disease in an individual by measuring the level of a biomarker in a biological sample, therefore, is not intended to be limiting and should not be construed that way.

The kit may comprise a negative control containing a biomarker at a concentration of about the concentration of the biomarker that is present in a biological sample of an individual who does not have Alzheimer's disease or does not have increased risk for Alzheimer's disease. The kit may also include a positive control containing the biomarker at a concentration of about the concentration of the biomarker that is present in a biological sample of an individual who has Alzheimer's disease or has increased risk for Alzheimer's disease.

Additionally, the kit includes at least the biomarker of the disclosure. Indeed, the disclosure should not be limited to only the marker disclosed herein because a skilled artisan, when aimed with the disclosure, would be able identify additional markers that can be used as indicators for Alzheimer's disease.

In another aspect, other factors that predict for AD can be included in the kit. Such factors include, but are not limited to, ApoE genotype (e2/e2, e2/e3, e2/e4, e3/e3, e3/e4 and e4/e4).

The kit of the disclosure can be used to assess the status of Alzheimer's disease in an individual, e.g., to diagnose Alzheimer's disease or to assess the degree of Alzheimer's disease in the individual. The phrase “Alzheimer's disease status” includes any distinguishable manifestation of the disease, including non-Alzheimer's disease, e.g., normal or non-demented. For example, disease status includes, without limitation, the presence or absence of Alzheimer's disease (e.g., Alzheimer's disease v. non-Alzheimer's disease), the risk of developing disease, the stage of the disease, the progress of disease (e.g., progress of disease or remission of disease over time), and the effectiveness or response to treatment of disease. Based on this status, further procedures may be indicated, including additional diagnostic tests or therapeutic procedures or regimens.

Furthermore, the kit includes an instructional material for use in the diagnosis of Alzheimer's disease in an individual. The instructional material can be a publication, a recording, a diagram, or any other medium of expression that can be used to communicate the usefulness of the method of the disclosure in the kit for assessment of Alzheimer's disease risk in an individual. The instructional material of the kit of the disclosure may, for example, be affixed to a container that contains other contents of the kit, or be shipped together with a container that contains the kit. Alternatively, the instructional material may be shipped separately from the container with the intention that the instructional material and the contents of the kit be used cooperatively by the recipient.

### Screening Methods for Compounds to Treat Alzheimer's Disease

In yet another embodiment, the disclosure provides a method of screening for a candidate compound for treating or preventing Alzheimer's disease, the method comprising the steps of a) contacting a test compound with a polypeptide encoded by TSPAN6, b) detecting binding activity between the polypeptide and the test compound or detecting biological activity of the polypeptide of step a), and c) selecting a compound that binds to the polypeptide or selecting a compound that suppresses biological activity of the polypeptide in comparison with the biological activity in the absence of the test compound.

In a specific embodiment, the disclosure provides screening methods for isolating agents that down-regulate the biological function of TSPAN6. In the context of the disclosure, agents to be identified through the screening methods can be any compound or composition. Furthermore, the test agent or compound exposed to a cell or protein according to the screening methods of the disclosure can be a single compound or a combination of compounds. When a combination of compounds is used in the methods, the compounds can be contacted sequentially or simultaneously. Any test agent or compound, for example, cell extracts, cell culture supernatant, products of fermenting microorganism, extracts from marine organism, plant extracts, purified or crude proteins, peptides, non-peptide compounds, synthetic micro-molecular compounds (including nucleic acid constructs, for example, antisense DNA, siRNA, ribozymes, etc.) and natural compounds can be used in the screening methods of the disclosure. The test agent or compound of the disclosure can also be obtained using any of the numerous approaches in combinatorial library methods known in the art, including (1) biological libraries, (2) spatially addressable parallel solid phase or solution phase libraries, (3) synthetic library methods requiring deconvolution, (4) the “one-bead one-compound” library method and (5) synthetic library methods using affinity chromatography selection. The biological library methods using affinity chromatography selection is limited to peptide libraries, while the other four approaches are applicable to peptide, non-peptide oligomer or small molecule libraries of compounds (Lam (1997) Anticancer Drug Design 12:145-67). Numerous examples of methods for the synthesis of molecular libraries can be found in the art. Libraries of compounds can be presented in solution or on beads, chips, bacteria, spores, plasmids or phage. A compound in which a part of the structure of the compound screened by any of the screening methods is converted by addition, deletion and/or replacement, and is included in the agents obtained by the screening methods of the disclosure. Furthermore, when the screened test agent or compound is a protein for obtaining a DNA encoding the protein, either the whole amino acid sequence of the protein can be determined to deduce the nucleic acid sequence coding for the protein, or partial amino acid sequence of the obtained protein can be analyzed to prepare an oligo DNA as a probe based on the sequence, and screen cDNA libraries with the probe to obtain a DNA encoding the protein. The obtained DNA finds use in preparing the test agent or compound, which is a candidate for treating or preventing neurodegenerative diseases such as Alzheimer's disease. Test agents or compounds useful in the screening described herein can also be antibodies or non-antibody binding proteins that specifically bind to one of the two extracellular parts of TSPAN6 protein or partial TSPAN6 peptides to prevent the dimerization of TSPAN6.

Once an inhibitor of the TSPAN6 activity has been identified, combinatorial chemistry techniques can be employed to construct any number of variants based on the chemical structure of the identified inhibitor. The resulting library of candidate inhibitors, or “test agents or compounds,” can be screened using the methods of the disclosure to identify test agents or compounds of the library that disrupt the TSPAN6 biological activity. Compounds that bind to TSPAN6 protein can be screened, for example, by immunoprecipitation. In immunoprecipitation, an immune complex is formed by adding antibodies or non-antibody binding proteins to a cell lysate prepared using an appropriate detergent. The immune complex consists of a polypeptide, a polypeptide having a binding affinity for the polypeptide, and an antibody or non-antibody binding protein.

Immunoprecipitation can also be conducted using antibodies against a polypeptide, in addition to using antibodies against the above epitopes, which antibodies can be prepared as described before. An immune complex can be precipitated, for example, by Protein A sepharose or Protein G sepharose when the antibody is a mouse IgG antibody. If the polypeptide of the disclosure is prepared as a fusion protein with an epitope, for example, GST, an immune complex can be formed in the same manner as in the use of the antibody against the polypeptide, using a substance specifically binding to these epitopes, for example, glutathione-Sepharose 4B. Immunoprecipitation can be performed by well-known methods described in the art. SDS-PAGE is commonly used for analysis of immunoprecipitated proteins and the bound protein can be analyzed by the molecular weight of the protein using gels with an appropriate concentration.

Since the protein bound to the polypeptide is difficult to detect by a common staining method, for example, Coomassie staining or silver staining, the detection sensitivity for the protein can be improved by culturing cells in culture medium containing radioactive isotope, “S-methionine or S cysteine,” labeling proteins in the cells, and detecting the proteins. The target protein can be purified directly from the SDS-polyacrylamide gel and its sequence can be determined, when the molecular weight of a protein has been revealed. As a method for screening for proteins that bind to the TSPAN6 polypeptide using the polypeptide, for example, West-Western blotting analysis can be used. Specifically, a protein binding to the TSPAN6 polypeptide can be obtained by preparing a cDNA library from cells, tissues, organs, or cultured cells expected to express a protein binding to the TSPAN6 polypeptide using a phage vector (e.g., ZAP), expressing the protein on LB-agarose, fixing the protein expressed on a filter, reacting the purified and labeled TSPAN6 polypeptide with the above filter, and detecting the plaques expressing proteins bound to the TSPAN6 polypeptide according to the label. The TSPAN6 polypeptide can be labeled by utilizing the binding between biotin and avidin, or by utilizing an antibody that specifically binds to the TSPAN6 polypeptide, or a peptide or polypeptide (for example, GST) that is fused to the TSPAN6 polypeptide.

Methods using radioisotope or fluorescence and such can be also used. The terms “label” and “detectable label” are used herein to refer to any composition detectable by spectroscopic, photochemical, biochemical, immunochemical, electrical, optical or chemical means. Such labels include biotin for staining with labeled streptavidin conjugate, magnetic beads (e.g., DYNABEADS™), fluorescent dyes (e.g., fluorescein, Texas red, rhodamine, green fluorescent protein, fluorescein isothiocyanate (FITC), and the like), radiolabels, enzymes (e.g., horse radish peroxidase, alkaline phosphatase, beta-galactosidase, beta-glucosidase, and others commonly used in an ELISA), and calorimetric labels, for example, colloidal gold or colored glass or plastic (e.g., polystyrene, polypropylene, latex, etc.) beads. Means of detecting such labels are well known to those of skill in the art. Thus, for example, radiolabels can be detected using photographic film or scintillation counters; fluorescent markers can be detected using a photodetector to detect emitted light. Enzymatic labels are typically detected by providing the enzyme with a substrate and detecting the reaction product produced by the action of the enzyme on the substrate, and calorimetric labels are detected by simply visualizing the colored label.

Alternatively, in another embodiment of the screening method of the disclosure, a two-hybrid system utilizing cells can be used. In the two-hybrid system, the polypeptide of the disclosure is fused to the GAL4-binding region and expressed in yeast cells. A cDNA library is prepared from cells expected to express a protein binding to the polypeptide of the disclosure, such that the library, when expressed, is fused to the VP16 or GAL4 transcriptional activation region. The cDNA library is then introduced into the above yeast cells and the cDNA derived from the library is isolated from the positive clones detected (when a protein binding to the polypeptide of the disclosure is expressed in yeast cells, the binding of the two activates a reporter gene, making positive clones detectable).

A protein encoded by the cDNA can be prepared by introducing the cDNA isolated above to E. coli and expressing the protein. A compound binding to TSPAN6 polypeptide can also be screened using affinity chromatography. For example, the TSPAN6 polypeptide can be immobilized on a carrier of an affinity column, and a test compound, containing a protein capable of binding to the TSPAN6 polypeptide, is applied to the column. A test compound herein can be, for example, cell extracts, cell lysates, etc. After loading the test compound, the column is washed, and compounds bound to the TSPAN6 polypeptide can be prepared. When the test compound is a protein, the amino acid sequence of the obtained protein is analyzed, an oligo DNA is synthesized based on the sequence, and cDNA libraries are screened using the oligo DNA as a probe to obtain a DNA encoding the protein.

A biosensor using the surface plasmon resonance phenomenon can be used as a means for detecting or quantifying the bound compound in the disclosure. When such a biosensor is used, the interaction between the TSPAN6 polypeptide and a test compound can be observed real-time as a surface plasmon resonance signal, using only a minute amount of polypeptide and without labeling (for example, BIAcore, Pharmacia). Therefore, it is possible to evaluate the binding between the TSPAN6 polypeptide and a test compound using a biosensor, for example, BIAcore.

As a method of screening for compounds that inhibit the binding between a TSPAN6 protein and a binding partner thereof (e.g., gamma-secretase), many methods well known by one skilled in the art can be used. For example, screening can be carried out as an in vitro assay system, for example, a cellular system. More specifically, first, either the TSPAN6 protein or the binding partner thereof is bound to a support, and the other protein is added together with a test compound thereto. Next, the mixture is incubated, washed and the other protein bound to the support is detected and/or measured. Promising candidate compounds can inhibit the binding between the TSPAN6 polypeptide and the above-mentioned binding partner. The binding between the TSPAN6 polypeptide and the above-mentioned binding partner can be detected or measured using antibodies to TSPAN6 or the binding partner. For example, after contacting, a binding partner is immobilized on a support with a test compound, and TSPAN6 is added, incubated and washed, and detection or measurement can be conducted using an antibody against TSPAN6 polypeptide.

Alternatively, TSPAN6 polypeptide may be immobilized on a support, and an antibody against a binding partner may be used for detection or measurement. In the case of using an antibody in the screening, the antibody is preferably labeled with one of the labeling substances mentioned in this specification, and detected or measured based on the labeling substance. Alternatively, the antibody against TSPAN6 or a binding partner may be used as a primary antibody to be detected with a secondary antibody that is labeled with a labeling substance. Furthermore, the antibody bound to the protein in the screening of the disclosure may be detected or measured using the protein G or protein A column. Furthermore, the production of amyloid beta can be determined according to any method known in the art. For example, a test compound is contacted with the polypeptide expressing cell, the cell is incubated for a sufficient time to allow production of amyloid beta, and then, the amount of amyloid beta can be detected.

Alternatively, a test compound is contacted with the polypeptide in vitro, the polypeptide is incubated under condition that allows production of amyloid beta, and then, the amount of amyloid beta can be detected. Furthermore, the expression level of a polypeptide or functional equivalent thereof can be detected according to any method known in the art. For example, a reporter assay can be used. Suitable reporter genes and host cells are well known in the art. The reporter construct required for the screening can be prepared by using the transcriptional regulatory region of the TSPAN6 gene or the downstream gene thereof.

When the transcriptional regulatory region of the gene has been known to those skilled in the art, a reporter construct can be prepared by using the previous sequence information. When the transcriptional regulatory region remains unidentified, a nucleotide segment containing the transcriptional regulatory region can be isolated from a genome library based on the nucleotide sequence information of the gene. Specifically, the reporter construct required for the screening can be prepared by connecting reporter gene sequence to the transcriptional regulatory region of a TSPAN6 gene of interest. The transcriptional regulatory region of a TSPAN6 gene is the region from a start codon to at least 500 bp upstream, for example, 1000 bp, for example, 5000 or 10000 bp upstream. A nucleotide segment containing the transcriptional regulatory region can be isolated from a genome library or can be propagated by PCR. Methods for identifying a transcriptional regulatory region, and also assay protocol are well known (Sambrook and Russell, Molecular Cloning: A Laboratory Manual, 3rd Ed., Chapter 17, 2001, Cold Springs Harbor Laboratory Press).

In the disclosure herein, over-expression of TSPAN6 in Alzheimer's disease was detected in specific brain regions and the over-expression was correlated with the Braak stages of the disease. Therefore, using the TSPAN6 gene, proteins encoded by the gene or transcriptional regulatory region of the gene, compounds can be screened that alter the expression of the gene or the biological activity of a polypeptide encoded by the gene. Such compounds can be used as pharmaceuticals for treating or preventing Alzheimer's disease or detecting agents for diagnosing Alzheimer's disease and assessing a prognosis of an Alzheimer's disease patient.

Specifically, the disclosure provides the method of screening for an agent or compound useful in diagnosing, treating or preventing cancers using the TSPAN6 polypeptide. An embodiment of this screening method includes the steps of: (a) contacting a test agent or compound with a polypeptide selected from the group consisting of TSPAN6 protein, or fragment thereof; (b) detecting binding between the polypeptide and the test agent or compound; and (c) selecting the test agent or compound that binds to the polypeptides of step (a). As a method of screening for proteins, for example, that bind to TSPAN6 polypeptide using TSPAN6 polypeptide, many methods well known by a person skilled in the art can be used. Such a screening can be conducted by, for example, an immunoprecipitation method. In a specific embodiment, a screening assay is provided for compounds that suppress the biological activity of the TSPAN6 gene. In the disclosure, the TSPAN6 protein has the activity of modulating the activity of gamma-secretase, which activity can be determined by the production of amyloid beta (i.e., the production of Abeta40 and the production of Abeta42). Using this biological activity, a compound that inhibits this activity of TSPAN6 can be screened. Therefore, the disclosure provides a method of screening for a compound for treating or preventing Alzheimer's disease, i.e., neurons overexpressing the TSPAN6 gene.

The term “suppress the biological activity” as defined herein refers to at least 10% suppression of the biological activity of TSPAN6 in comparison with an absence of the compound, for example, at least 25%, 50% or 75% suppression, for example, at least 90% suppression.

Cells expressing the TSPAN6 include, for example, cell lines (e.g., neuron or neuronal cell lines) that can be generated; such cells can be used for the above screening of the disclosure. The expression level can be estimated by methods well known to one skilled in the art, for example, RT-PCR, Northern blot assay, Western blot assay, immunostaining, ELISA or flow cytometry analysis. The term “reduce the expression level” as defined herein refers to at least 10% reduction of expression level of TSPAN6 in comparison to the expression level in absence of the compound, for example, at least 25%, 50% or 75% reduced level, for example, at least 95% reduced level. The compound herein includes chemical compound, double-strand nucleotide, and so on. The preparation of the double-strand nucleotide is in the aforementioned description. In the method of screening, a compound that reduces the expression level of TSPAN6 can be selected as candidate agents or compounds to be used for the treatment or prevention of Alzheimer's disease.

Alternatively, the screening method of the disclosure can include the following steps: a) contacting a candidate compound with a cell into which a vector, including the transcriptional regulatory region of TSPAN6 and a reporter gene that is expressed under the control of the transcriptional regulatory region, has been introduced, b) measuring the expression or activity of the reporter gene, and c) selecting the candidate compound that reduces the expression or activity of the reporter gene. Suitable reporter genes and host cells are well known in the art. For example, reporter genes are luciferase, green florescence protein (GFP), Red fluorescent protein (RFP), Chloramphenicol Acetyltransferase (CAT), lacZ and beta-glucuronidase (GUS), and a host cell is, for example, COS7, HEK293, HeLa and so on.

Aspects of the disclosure are described in the following examples, which are not intended to limit the scope of the disclosure described in the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the disclosure, suitable methods and materials are described below.

### Examples

### 1. Expression of TSPAN6 in AD Patients

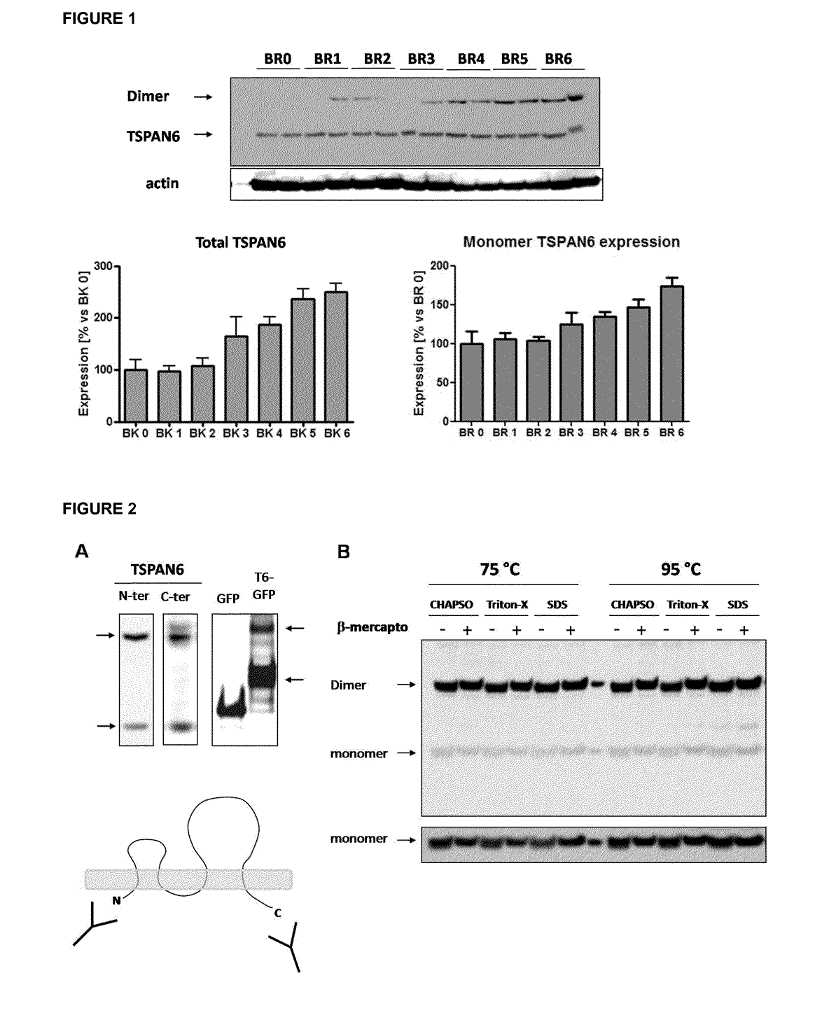

In the disclosure, we investigated the changes in the expression of genes belonging to the tetraspanin family during the clinical evolution of the AD pathology. A positive correlation was identified between the mRNA levels of Tetraspanin-6 (TSPAN6) in the cerebral prefrontal cortex and surprisingly also with the Braak stages of the disease (Bossers et al., 2010). In the next step, we investigated the protein expression of TSPAN6 in the same samples. These data showed that the same positive correlation was observed for the protein expression as detected in a Western blot with a commercial polyclonal anti-TSPAN6 antibody (Abeam) after running a 4-12% Bis-Tris gel (FIG. 1). In addition, a band corresponding to the molecular weight of a putative dimer (50 kDa) was detected, which also followed a positive linear correlation with the Braak stages of the disease.

Tetraspanins have been described to form functional homodimers and heterodimers between other tetraspanins members of the family (Kovalenko et al., 2004; Bari et al., 2009; Kitadokoro et al., 2001; Seigneuret et al., 2001), as well as homotrimers and homotetramers (reviewed in Zoller, 2009). To determine if the 50 kDa band corresponds to the dimer of TSPAN6, we used two different polyclonal antibodies against the TSPAN6 protein on a Western blot, one with a specificity for the N-terminus and the other with a specificity for the C-terminus. Both antibodies gave two bands on the nitrocellulose membrane, one with an apparent molecular weight of about 28 kDa and the other at about 50 kDa. On the other hand, when we overexpressed TSPAN6 fused with GFP at the C-terminus of the protein in HEK cells and ran the sample in a 4-12% BisTris gel, a band appeared corresponding to the approximate molecular weight of two times the TSPAN6-GFP fused protein (FIG. 2, Panel A). In order to determine if the dimer is covalently formed, we exposed lysates from HEK cells to strong (1% SDS) or milder (1% CHAPSO, 1% TRITON®-X-100) detergents, high temperatures (95° C.) and to the presence or the absence of a reducing agent (β-mercaptoethanol). Since none of the treatments managed to disrupt the band corresponding to the dimer, we concluded that it is covalently formed (see FIG. 2, Panel B). Due to the absence of data about TSPAN6 in the scientific literature regarding its localization, expression, and function, we studied its expression by Western blot in the mouse brain and in neurons by using a commercial polyclonal antibody against the N-terminus of the protein (Abeam), as well as the expression of TSPAN6 during brain development.

### 2. Expression and Localization of TSPAN6 in the Brain

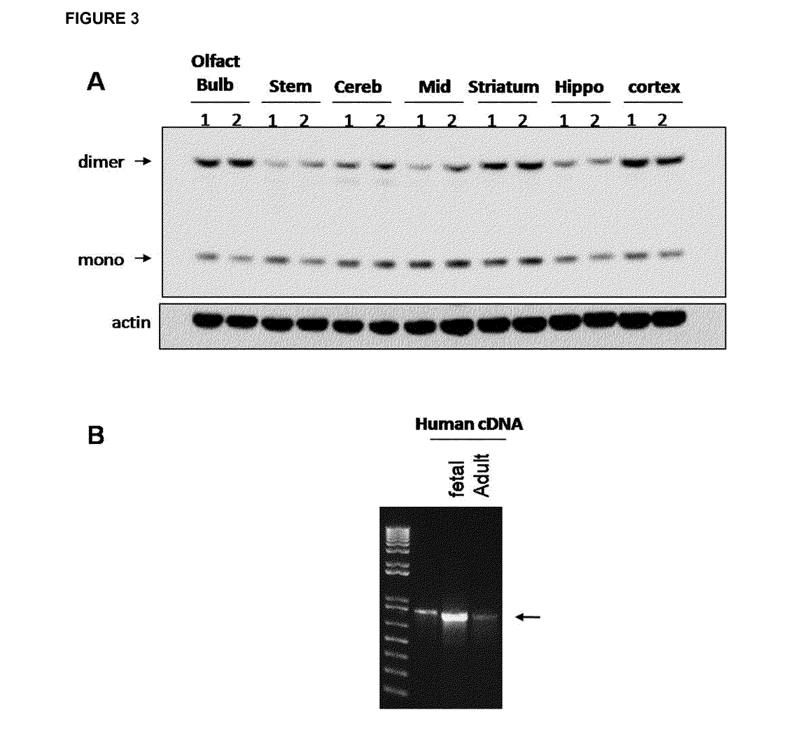

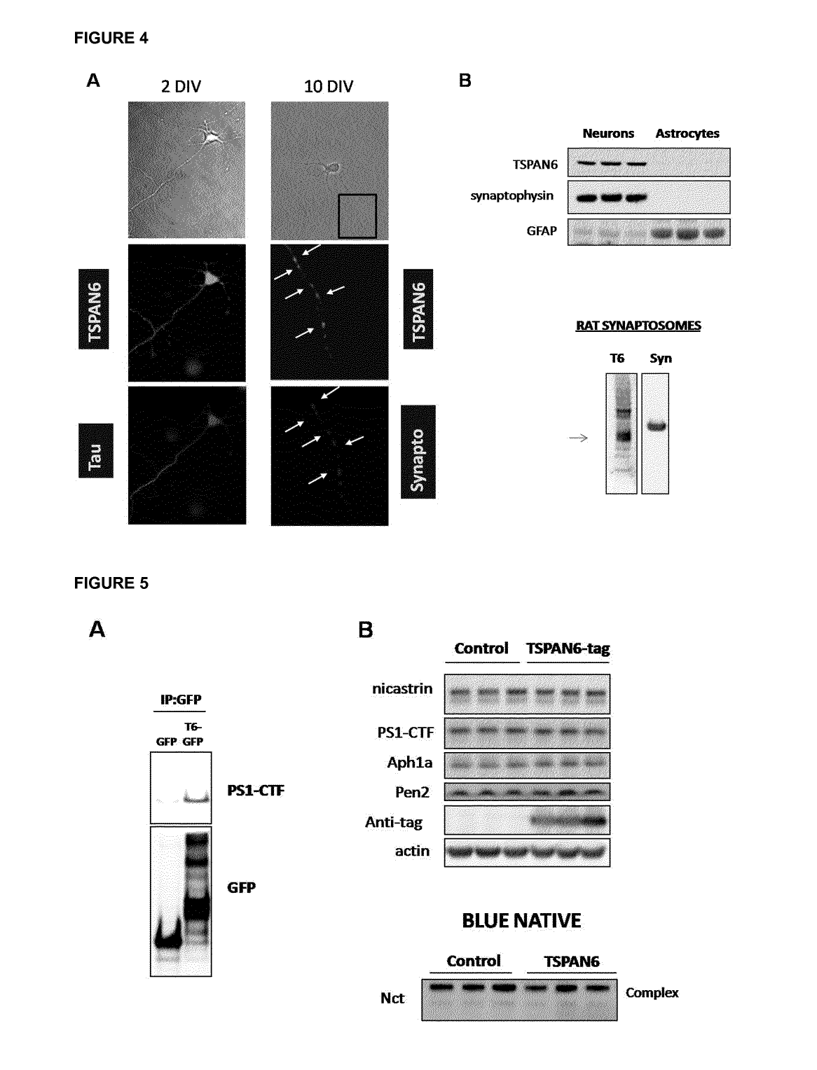

We showed that TSPAN6 appears to be widely expressed in several regions of the brain and particularly also in brain areas that are predominantly affected in AD (i.e., the cortex and hippocampus) (see FIG. 3, Panel A). By PCR of the total human cDNA from the cortical region of the brain, it was shown that the total mRNA for TSPAN6 is higher during development (i.e., in fetal brain) as compared to the adult brain (see FIG. 3, Panel B). TSPAN6 is highly expressed in rat primary hippocampal neurons after only 10 days in vitro (DIV), while no detection was found in rat astroglial cultures (see FIG. 4, Panel A). By using immunofluorescence to study the localization of the TSPAN6 protein 2 DIV on rat primary hippocampal neurons, the protein shows a predominant axonal localization (tau is used as an axonal marker) from the very early stages of neuronal development in vitro (see FIG. 4, Panel B). In mature neurons (10 DIV) it colocalizes with synaptophysin, an axonal pre-synaptic protein (see FIG. 4, Panel B). In order to study the presence of TSPAN6 in the neuronal synapsis, we prepared synaptosomes from adult rats. After running the samples in a 4-12% BisTris gel and transferring the samples onto a nitrocellulose membrane, we detected TSPAN6 in this fraction (FIG. 4, Panel B).

### 3. Interaction of TSPAN6 with γ-Secretase and Regulation of Aβ Production

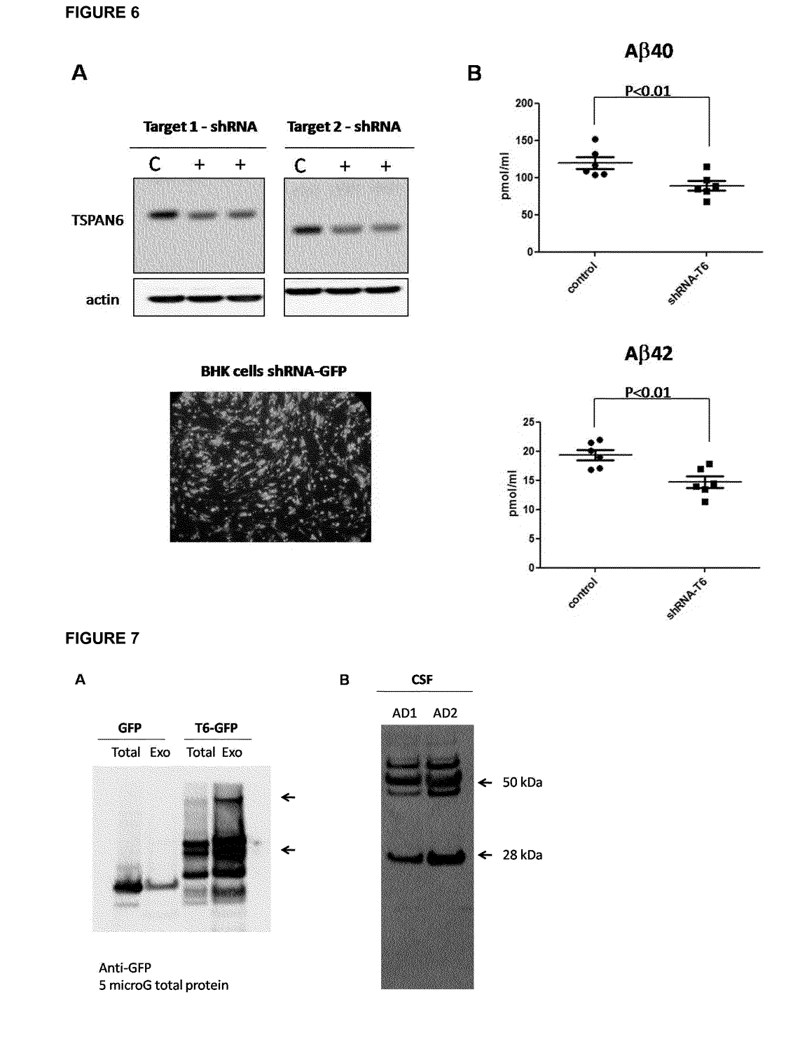

Since tetraspanins (CD81 and CD9) were reported to interact directly with PS1 and thereby modulate the γ-secretase function (Wakabayashi et al., 2009), we investigated if TSPAN6 was also interacting with PS1. For this, we overexpressed either the TSPAN6-GFP fusion protein or only GFP in HEK cells for 48 hours. After lysing the cells in lysis buffer containing 1% TRITON®-X-100 detergent, an anti-GFP nanobody covalently bound to beads was added to the samples. After overnight (o/n) incubation at 4° C., the beads were immunoprecipitated and the proteins were separated from the beads by boiling them in reducing conditions (5% β-mercaptoethanol). The samples were run in a 4-12% BisTris gel and transferred onto a nitrocellulose membrane. A monoclonal antibody against the C-terminal fragment of presenilin 1 (PS1-CTF) and an anti-GFP polyclonal antibody were used to detect co-precipitated PS1 and to check for the efficiency of the immunoprecipitation, respectively. As shown in FIG. 5, Panel A, PS1 co-immunoprecipitated with TSPAN6-GFP but not with GFP alone, is pointing out to a direct interaction between PS and TSPAN6. No major changes in the expression of PS or other components of the γ-secretase complex were observed by Western blot (see FIG. 5, Panel B), meaning that the increased co-precipitation was not due to the presence of more γ-secretase. A Blue Native gel was run with lysates from HEK transfected or untransfected with TSPAN6. No differences were observed regarding the total complex assembly in cells overexpressing TSPAN6 (see FIG. 5, Panel B). In the next step, we studied the effect of TSPAN6 down-regulation on the generation of amyloid beta (Abeta). We designed two distinct shRNA sequences against the rat TSPAN6 mRNA (5′-TTCATCTTTTGGATCACTG-3′ (SEQ ID NO:6) and 5′-CAGACATGAGATTAAGAAC-3′) (SEQ ID NO:7), which were tested in a hamster cell line (BHK cells) 48 hours after transfection. The vector (pA6P-CAG-EGFP) included the EGFP reporter to follow the efficiency of transfection. After running a 4-12% BisTris gel with lysates from non-transfected or transfected BHK cells, the down-regulation of the protein was evaluated by Western blot in combination with the use of a commercial polyclonal anti-TSPAN6 antibody (Abcam) (see FIG. 6, Panel A). After confirming the efficacy of our shRNA constructs on BHK cells, we proceeded to study the effect on Aβ generation in a rat primary hippocampal culture. For this purpose, we transfected primary neurons in suspension with the non-viral nucleofector kit AMAXA (Lonza Cologne, Germany) at day 0 before seeding them on 6-well poly-lysinated dishes (150,000 neurons per well) in B27-supplemented neurobasal media (Gibso). After 8 DIV in vitro, the media was analyzed for Aβ species with an in-house ELISA sandwich. Briefly, 96-wells Nunc-Immuno plates (Nunc, Denmark) were coated overnight at 4° C. with JRF cAb040/28 antibody for Aβ40 or JRF Ab042/26 antibody for Aβ42 (Janssen Pharmaceutica), both used at 1.5 mg/ml in PBS containing 0.1% casein (Casein Buffer). Plates were washed five times with Washing Buffer (PBS-0.05% TWEEN® 20) before the addition of the samples or the standard curve made with consecutive dilutions (from 100 to 0.0003 ng/ml) of Aβ40 or Aβ42 (rPeptide). After overnight incubation at 4° C. and five times washes with the Washing Buffer, the samples were developed with a 0.02% TMB (tetramethylbenzidine) solution in Sodium Acetate (100 mM pH 4.9) containing 0.03% H₂O₂. The reaction was stopped with 0.2 N H₂SO₄ and read at 450 nm. The results of the measurements, as shown in FIG. 6, Panel B, indicate a decrease in Aβ40 and Aβ42 secretion when the expression of TSPAN6 is down-regulated.

These data convincingly show that TSPAN6 is a new potential therapeutic target for AD. Furthermore, our results demonstrate that TSPAN6 is a neuronal protein, mainly localized in axons, which levels increase during the clinical evolution of sporadic AD. TSPAN6 interacts with PS1 and its down-regulation by shRNA in rat primary hippocampal neurons decreases the production of both Aβ₄₀ and Aβ₄₂. The use of monoclonal antibodies against TSPAN6 could control the Aβ generation by disrupting the interaction with PS1.

### 4. Secretion of TSPAN6 in Exosomes and its Presence in Cerebrospinal Fluid (CSF)

In the next step, we investigated the use of TSPAN6 as a biomarker to follow up the evolution of AD in patients. It is described in the art that many tetraspanins are present in exosomes, formed in late-endosomes as multilamellar bodies and secreted upon fusion with the plasma membrane. Exosomes are lipoprotein structures of about 50-100 nm diameter and enriched with certain proteins, lipids and nucleic acids. They are thought to serve as a system of cell-to-cell communication and can modulate the gene expression of other cells by loading the host cell with microRNAs. Since exosomes are found in several biological fluids and manage to go through the BBB, molecules found in exosomes have been proposed as possible biomarkers for many diseases. For this reason, we investigated if TSPAN6 is present in exosomes. We overexpressed TSPAN6-GFP or GFP alone in HEK 293T cells in two T175 flasks each (9,300,000 cells/flask). After 24 hours, we changed the media for exosomes-depleted media (obtained by centrifugation at 100,000 g o/n at 4° C.) and incubated the cells for 24 hours. The exosomal fraction was obtained by a discontinuous sucrose gradient by ultracentrifugation overnight at 4° C. (100,000 rpm) and after washing the exosomes with PBS, they were recovered by centrifugation at 55,000 rpm for 1 hour at 4° C. The samples (total lysates from GFP and TSPAN6 overexpressing HEK cells or exosomal fractions and their media) were processed for Western blotting and the membrane developed with a polyclonal anti-GFP antibody. The results depicted in FIG. 7, Panel A show an enrichment of TSPAN6-GFP in the exosomal fraction when compared to the lane corresponding to the total lysate. On the contrary, the GFP alone is enriched in the total lysate (see FIG. 7, Panel A).

Thus, our findings show that tetraspanin-6 is secreted in exosomes, meaning that it could be found in many biological fluids like the cerebrospinal fluid (CSF), plasma, saliva or urine. Indeed independent reports of the literature indicate that TSPAN6 has been found in the saliva and the urine in two independent proteomic studies (Gonzalez-Begne et al., 2009; Gonzalez et al., 2009). We checked for the presence of TSPAN6 in the CSF from two AD patients. The samples (25 μL) were run in a 4-12% BisTris gel and transferred onto a nitrocellulose membrane for detection of the protein with a polyclonal antibody against the C-terminus of the protein. As shown in FIG. 7, we could successfully detect TSPAN6 in both CSF samples.

### 5. Effect of Down-Regulating TSPAN6 on Other γ-Secretase Substrates

In the next step, we studied the effect of the down-regulation of TSPAN6 on the processing of other reported gamma-secretase substrates. Accordingly, we checked for the gamma-secretase activity on APP-C99, Notch, Syndecan-3, ADAM10, Neuroregulin and E-cadherin. For the processing of APP-C99 and Notch, HEK293 cells are co-transfected with the UAS-luciferase reporter gene, an APP or Notch reporter construct carrying a Gal4-VP16 (Serneels et al., 2005) in the cytoplasmic domain, and specific siRNA oligonucleotides targeting TSPAN6 are used for down-regulating the TSPAN6 activity. After 48 hours, the cells are lysed and processed according to the manufacturer's instructions (Promega, Leiden, Netherlands), and emitted light is measured with the microplate reader (Victor3 by PerkinElmer, Zaventem, Belgium). For the other substrates, after transfecting HEK293 cells with the pA6P-CAG-EGFP-shRNA construct against TSPAN6 for 48 hours, the cells are lysated in lysis buffer containing 1% TRITON®-X-100 and run in a 4-12% BisTris gel. The gel is transferred onto a nitrocellulose membrane to evaluate the levels of the gamma-secretase-dependent C-terminal fragments (CTF) by using specific antibodies against Neuroregulin, Syndecan-3 and ADAM10.

### 6. In Vivo Validation of the Effect of Down-Regulating TSPAN6 on Aβ Production

We are creating an adeno-associated virus (AAV) using the pA6P-CAG-EGFP-shRNA construct (AAV-EGFP-shRNA/TSPAN6) to down-regulate in vivo the expression of TSPAN6 by stereotactical injection of the recombinant virus into the brain of mice. We are also creating an AAV expressing a scrambled shRNA that does not match any mammalian mRNA (AAV-EGFP-shRNA) for use as a negative control. In addition, we are studying the effect of down-regulating TSPAN6 in one of the hippocampus of 1 year old White Swiss mice, while the other hippocampus is stereotactically injected with the AAV-EGFP-shRNA as a negative control. After two weeks, the hippocampus of six mice is isolated to quantify the amount of Aβ40 and Aβ42 by ELISA as described previously.

### 7. TSPAN6 as a Biomarker for AD

Since we have convincingly shown that the TSPAN6 protein levels are elevated during the Braak Stages of AD, we also checked the use of TSPAN6 as a clinical marker to follow the evolution of AD disease. First, we checked for the presence of TSPAN6 in several human biological fluids (CSF, saliva, plasma and urine). The samples (25 μL) are run in a 4-12% BisTris gel and transferred onto a nitrocellulose membrane for detection of the TSPAN6 protein with a polyclonal antibody against the C-terminus of the protein. Next, we evaluated by Western blot the differences in the levels of TSPAN6 between AD patients of several disease stages, healthy control individuals and individuals suffering from other neurodegenerative diseases (e.g., Parkinson disease, frontotemporal lobe dementia, Lewy-Body dementia, Huntington disease) in any of the biological fluids.

### 8. Effect of TSPAN6 Overexpression on Abeta Secretion by HEK-APPsw

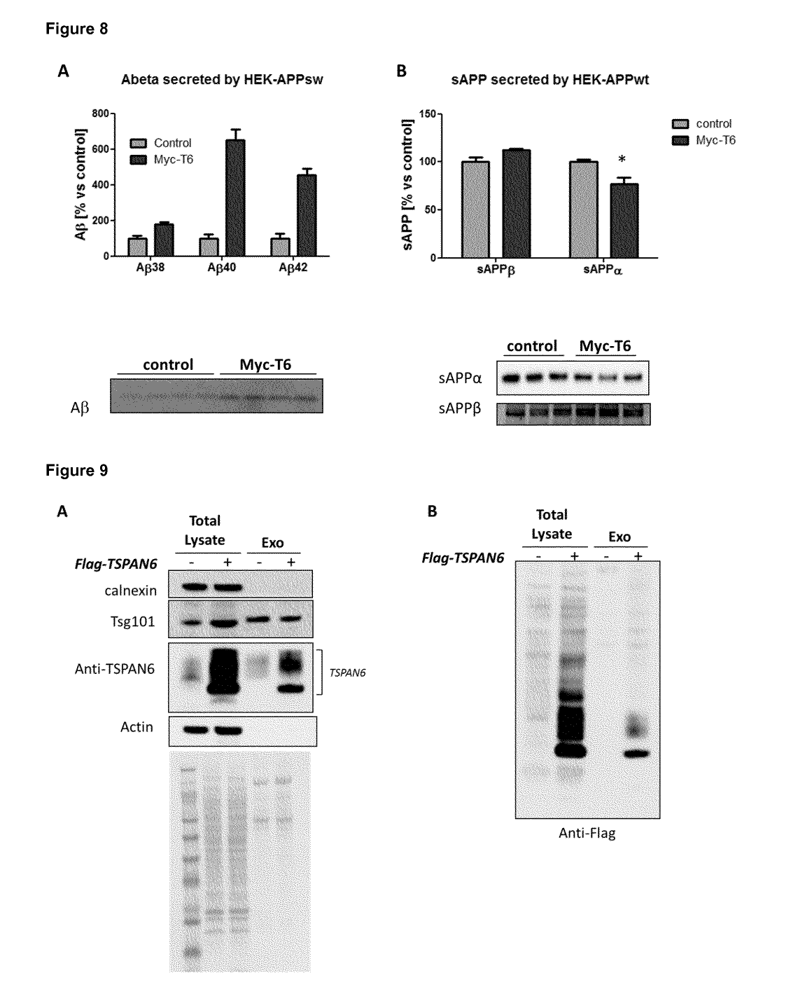

HEK cells stably expressing the APP Swedish mutant were transfected with a myc-TSPAN6 fusion. It is shown in FIG. 8 that overexpression of TSPAN6 increases the levels of Abeta species secreted into the medium of the cells. On the other hand, the effect of TSPAN6 overexpression on sAPPalpha (i.e., the non-toxic or protective fragment) secretion is minimal. These in vitro experiments show that an inhibitor of TSPAN6 would normalize the levels of Abeta while not influencing the sAPPalpha levels.

### 9. Detection of TSPAN6 in Exosomes

FIG. 9 shows that HEK293 cells transfected with a flag-tagged TSPAN6 are able to form exosomes that comprise the TSPAN6 protein.

### 10. Detection of TSPAN6 Protein in CSF Samples

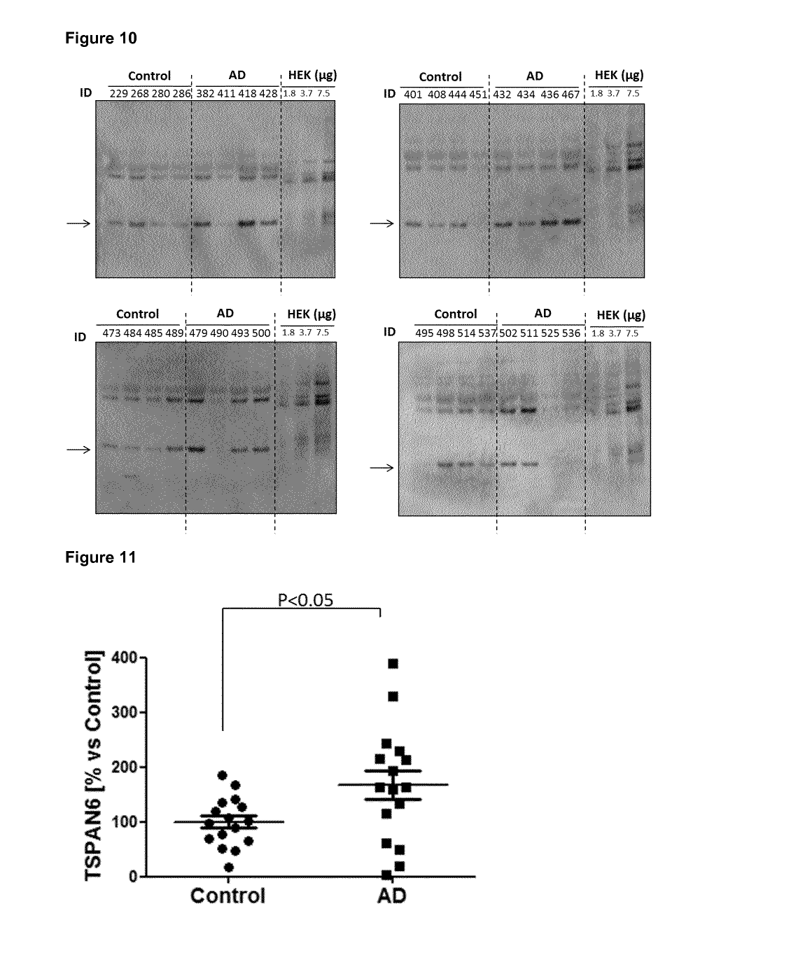

FIG. 10 shows the presence of TSPAN6 in CSF samples derived from controls and AD patients.

### 11. Comparison of the TSPAN6 Levels in CSF of AD Patients and Controls

FIG. 11 shows that the quantification of TSPAN6 levels in CSF can differentiate AD patients (n=16) and controls ((n=16).

### 12. Correlation Between the Levels of TSPAN6 and the INNOTEST® Amyloid Tau Index (IATI)

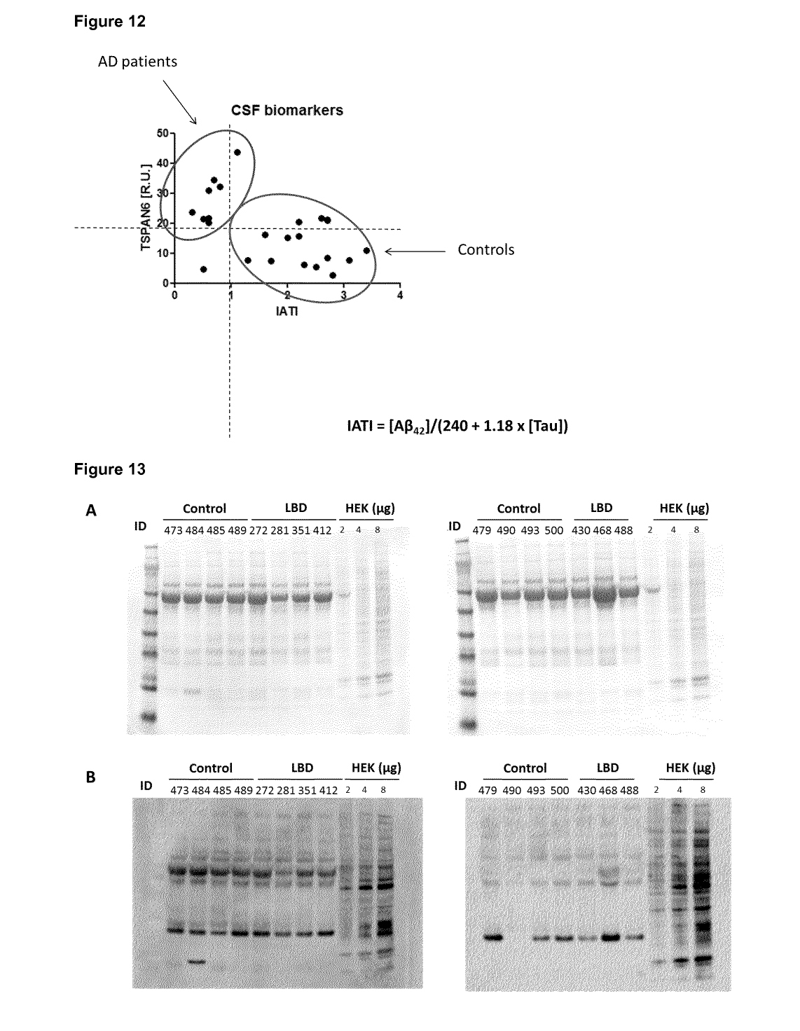

FIG. 12 shows a correlation between the TSPAN6 levels present in CSF and the determination of amyloid beta and tau (through the application of the INNOTEST® Amyloid Tau Index (IATI-test)). The IATI test is described in, for example, F. Tabaraud et al. (2012), Acta Neurol. Scand. 125:416-423. A control subject with normal Abeta1-42 and T-tau values has an IATI>1. A patient with possible AD, i.e., with a lowered Abeta1-42 and increased tau value, has an IATI<1.

### 13. Diagnostic Utility of TSPAN6 Determination for AD Disease

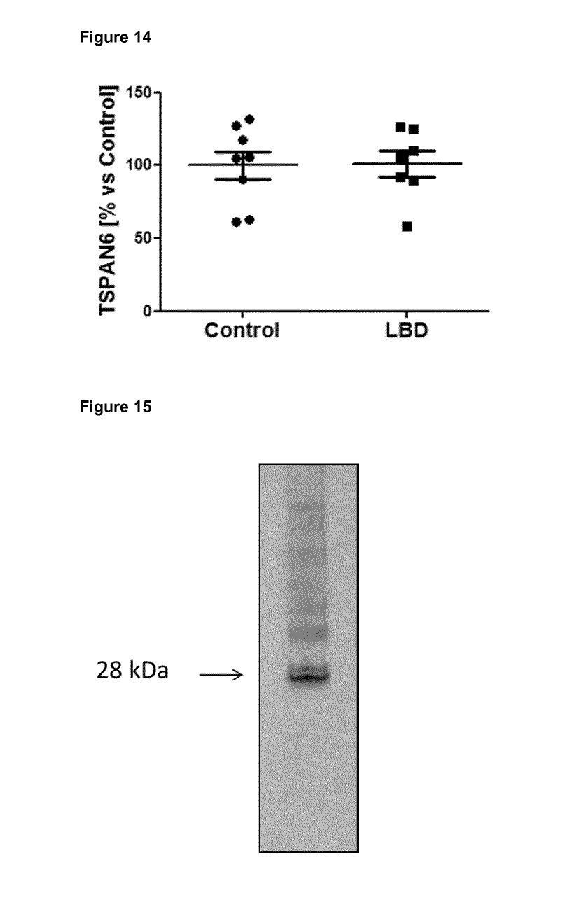

FIGS. 13 and 14 show that the determination of TSPAN6 levels cannot be used to differentiate controls and patients suffering from Lewy-Body dementia (LBD). We conclude that de-quantification of TSPAN6 is specific for the detection of Alzheimer's disease patients (see FIG. 11).

### 14. Detection of TSPAN6 in Saliva

FIG. 15 shows the presence of TSPAN6 in a saliva sample.

### Materials and Methods

### 1. Preparation of Cell Lysates and Western Blot

Total cell extracts were prepared in TBS (50 mM Tris-HCl pH 7.4, 150 mM NaCl) containing 1% TRITON®-X100, and Complete protease inhibitors (Roche Applied Science). Insoluble fractions were removed by centrifugation at 15,000×g for 15 minutes at 4° C. Protein concentration was determined by the Bradford dye-binding procedure (Bio-Rad). Proteins were separated on 4-12%, 10% or 12% NuPAGE® Bis-Tris gels (Invitrogen) and were transferred to nitrocellulose membranes. Membranes were blocked with 5% skim milk in TBS and probed with antibodies followed by incubation with horseradish peroxidase conjugated antibodies (Bio-Rad). Bands were detected with Renaissance (ParkinElmer).

### 2. Analysis of APP Processing

Twenty-four hours before transfection, HEK293 cells or hippocampal neurons stably expressing APP bearing Swedish mutation (KM670/671NL) were plated out in 24-well plates. The cells were transfected with ON-TARGET PLUS® SMARTpool or Duplex (for Ptgfrn, Igsf8, Itgb1, Itga3, Slc3a2, CD81, CD9 and ATP1A1) siRNAs (Dhaimacon) using LipofectAMINE2000 (Invitrogen). For control transfection, SiCONTROL™ Non-targeting pool siRNA was used. Thirty-two hours after transfection, medium was changed to DMEM supplemented with 1% FBS and 16 hours later, the medium was collected. The medium was centrifuged at 800×g for five minutes at 4° C. to remove cells. Supernatant was used in a specific ELISA to detect Aβ40 and Aβ42 (The Genetics Company) according to the manufacturer's instructions. For analysis in Hela cells, cells were plated in 24-well plates and the cells were transfected with siRNAs. Twenty hours later, the cells were infected with human APP-Swedish-695 (APP695Sw) adenovirus using an infection multiplicity of 50. After six hours of infection, the cells were rinsed once with DPBS and medium was changed to DMEM supplemented with 1% FBS. Sixteen hours later, the medium was collected and subjected to ELISA. Total cell extracts were prepared in lysis buffer (1% TRITON® X-100, 1% sodium deoxycholate, 0.1% SDS in HEPES buffer with complete protease inhibitors) and insoluble fractions were removed by centrifugation at 15,000×g for 15 minutes at 4° C. Equal amounts of proteins were separated by SDS-PAGE and detected by Western blot.

For the in vitro γ-secretase assay, samples were mixed with the recombinant substrate APP C99-FLAG purified from E. coli expressing C99-FLAG. After incubation at 37° C., de novo formed Aβ peptides were separated on 12% NuPAGE® Bis-Tris gels followed by Western blot.

### 3. Statistical Analysis

Data are presented as mean values and error bars indicate the standard error of the mean (SEM). The treatment groups were compared by one-way analysis of variance (ANOVA) using Dunnett's post hoc pair-wise multiple comparisons tests or two-tailed Student's t-test. Significance was set at *P<0.05; **P<0.01; and ***P<0.001. Statistical calculations were made using the PRISM version 4 statistical software (GraphPad Software).

### REFERENCES

Bari R., Y. H. Zhang, F. Zhang, N. X. Wang, C. S. Stipp, J. J. Zheng, and X. A. Zhang (2009). Transmembrane interactions are needed for KAI1/CD82-mediated suppression of cancer invasion and metastasis. Am. J. Pathol. 174:647-60.
Bossers K., K. T. Wirz, G. F. Meerhoff, A. H. Essing, J. W. van Dongen, P. Houba, C. G. Kruse, J. Verhaagen, and D. F. Swaab (2010). Concerted changes in transcripts in the prefrontal cortex precede neuropathology in Alzheimer's disease. Brain 133:3699-723.
De Strooper B. (2010). Proteases and proteolysis in Alzheimer disease: a multifactorial view on the disease process. Physiol. Rev. 90; 465-94.
De Strooper B., P. Saftig, K. Craessaerts, H. Vanderstichele, G. Guhde, W. Annaert, K. Von Figura, and F. Van Leuven (1998). Deficiency of presenilin-1 inhibits the normal cleavage of amyloid precursor protein. Nature 391; 387-90.
Gonzales P. A., T. Pisitkun, J. D. Hoffert, D. Tchapyjnikov, R. A. Star, R. Kleta, N. S. Wang, and M. A. Knepper. Large-scale proteomics and phosphoproteomics of urinary exosomes. J. Am. Soc. Nephrol. 20:363-79.
Gonzalez-Begne M., B. Lu, X. Han, F. K. Hagen, A. R. Hand, J. E. Melvin, and J. R. Yates (2009). Proteomic analysis of human parotid gland exosomes by multidimensional protein identification technology (MudPIT). J. Proteome Res. 8:1304-14.
Hardy J., and D. J. Selkoe (2002). The amyloid hypothesis of Alzheimer's disease: progress and problems on the road to therapeutics. Science 297:353-6.
Kovalenko O. V., X. Yang, T. V. Kolesnikova, and M. E. Hemler. Evidence for specific tetraspanin homodimers: inhibition of palmitoylation makes cysteine residues available for cross-linking. Biochem. J. 377:407-17.
Seigneuret M., A. Delaguillaumie, C. Lagaudrière-Gesbert, and H. Conjeaud (2001). Structure of the tetraspanin main extracellular domain. A partially conserved fold with a structurally variable domain insertion. J. Biol. Chem. 276:40055-64.
Serneels L., T. Dejaegere, K. Craessaerts, K. Horré, E. Jorissen, T. Tousseyn, S. Hébert, M. Coolen, G. Martens, A. Zwijsen, W. Annaert, D. Hartmann, and B. De Strooper (2005). Differential contribution of the three Aph1 genes to gamma-secretase activity in vivo. Proc. Natl. Acad. Sci. U.S.A. 102:1719-24.
Wakabayashi T., K. Craessaerts, L. Bammens, M. Bentahir, F. Borgions, P. Herdewijn, A. Staes, E. Timmerman, J. Vandekerckhove, E. Rubinstein, C. Boucheix, K. Gevaert, and B. De Strooper (2009). Analysis of the gamma-secretase interactome and validation of its association with tetraspanin-enriched microdomains. Nat. Cell Biol. 11:1340-6.
Zlokovic B. (2008). The blood-brain barrier in health and chronic neurodegenerative disorders. Neuron 57; 178-201
Zöller M. (2009). Tetraspanins: push and pull in suppressing and promoting metastasis. Nat. Rev. Cancer 9:40-55.

1. A method of detecting or diagnosing the presence of Alzheimer's disease or a predisposition to Alzheimer's disease in a subject, the method comprising:
determining the expression level of TSPAN6 in a biological sample derived from the subject, wherein an increase of said level compared to a normal control of said gene indicates that the subject suffers from or is at risk of developing Alzheimer's disease,
wherein the expression level is determined by any one method selected from the group consisting of: a) detecting a mRNA of TSPAN6, b) detecting a protein encoded by TSPAN6 and c) detecting the biological activity of the protein encoded by TSPAN6.

2. A method according to claim 1 wherein an increase of the level of TSPAN6 is correlated with the disease stage of Alzheimer's disease.

3. The method according to claim 1, wherein said increase is at least 10% greater than said normal control.

4. The method according to claim 1, wherein said biological sample is serum, plasma, saliva, CSF or urine.

5. A kit for detecting or diagnosing Alzheimer's disease in a subject, the kit comprising:
a detection agent that binds to a transcription or translation product of TSPAN6.

6. A method of treating or preventing Alzheimer's disease in a subject, the method comprising:
administering to the subject a compound that inhibits the biological activity of TSPAN6, the compound selected from the group consisting of a short interference RNA for TSPAN6, an antibody against TSPAN6 or a gene product thereof, and a peptide or an extracellular fragment derived from TSPAN6 so as to treat or prevent Alzheimer's disease in the subject.

7. A pharmaceutical composition comprising an effective amount of an isolated siRNA comprising a sense RNA strand and an antisense RNA strand, wherein the sense and the antisense RNA strands form an RNA duplex, and wherein the sense RNA strand comprises a nucleotide sequence identical to a target sequence of about 19 to about 25 contiguous nucleotides in SEQ ID NO:1.

8. A pharmaceutical composition comprising an effective amount of an antibody that specifically binds to SEQ ID NO:2.

9. A method of screening for a candidate compound for treating or preventing Alzheimer's disease, said method comprising the steps of:
a) contacting a test compound with a polypeptide encoded by TSPAN6,
b) detecting binding activity between the polypeptide and the test compound or detecting biological activity of the polypeptide of step a), and
c) selecting a compound that binds to the polypeptide or selecting a compound that suppresses biological activity of the polypeptide in comparison with the biological activity in the absence of the test compound.

10. A method of screening for a candidate compound for treating or preventing Alzheimer's disease, said method comprising the steps of:
a) contacting a test compound with a cell expressing TSPAN6, and
b) selecting a compound that reduces the expression level of TSPAN6.

11. A method of screening for a candidate compound for treating or preventing Alzheimer's disease, said method comprising:
contacting a test compound with a cell into which a vector comprising a transcriptional regulatory region of TSPAN6 gene and a reporter gene that is expressed under control of said transcriptional regulatory region has been introduced,
measuring expression or activity of said reporter gene, and
selecting a compound that reduces the expression or activity level of said reporter gene, as compared to a level in the absence of the test compound.

12. The method according to claim 2, wherein the increase is at least 10% greater than the normal control.

13. The method according to claim 2, wherein the biological sample is serum, plasma, saliva, cerebrospinal fluid, or urine.

14. The method according to claim 3, wherein the biological sample is serum, plasma, saliva, cerebrospinal fluid, or urine.

15. The method according to claim 12, wherein the biological sample is serum, plasma, saliva, cerebrospinal fluid, or urine.


SEQUENCE LISTING (7 sequences)

SEQ ID NO: 1 - DNA, 2036 bp, Homo sapiens
gggactccgcgtctcgctctctgtgttccaatcgcccggtgcggtggtgcagggtctcgggctagtcatggcgtccccgtctcggagactgcagactaaaccagtcattacttgtttcaagagcgttctgctaatctacacttttattttctggatcactggcgttatccttcttgcagttggcatttggggcaaggtgagcctggagaattacttttctcttttaaatgagaaggccaccaatgtccccttcgtgctaattgctactggtaccgtcattattcttttgggcacctttggttgttttgctacctgccgagcttctgcatggatgctaaaactgtatgcaatgtttctgactctcgtttttttggtcgaactggtcgctgccatcgtaggatttgttttcagacatgagattaagaacagctttaagaataattatgagaaggctttgaagcagtataactctacaggagattatagaagccatgcagtagacaagatccaaaatacgttgcattgttgtggtgtcaccgattatagagattggacagatactaattattactcagaaaaaggatttcctaagagttgctgtaaacttgaagattgtactccacagagagatgcagacaaagtaaacaatgaaggttgttttataaaggtgatgaccattatagagtcagaaatgggagtcgttgcaggaatttcctttggagttgcttgcttccaactgattggaatctttctcgcctactgcctctctcgtgccataacaaataaccagtatgagatagtgtaacccaatgtatctgtgggcctattcctctctacctttaaggacatttaggtcccccctgtgaattagaaagttgcttggctggagaactgacagcactacttactgatagaccaaaaaactacaccagtaggttgattcaatcaagatgtatgtagacctaaaactacaccaataggctgattcaatcaagatccgtgctcgcagtgggctgattcaatcaagatgtatgtttgctatgttctaagtccaccttctatcccattcatgttagatcgttgaaacctggtctccctctgaaacactggaagagctagtaaattgtaaatgaagtaatactgtgttcctcttgactgttatttttcttagtagggggcctttggaaggcactgtgaatttgctattttgatgtagtgttaccaagatggaaaattgattcctctgactttgctattgatgtagtgtgatagaaaattcacccctctgaactggctccttcccagtcaaggttatctggtttgattgtataatttgcaccaagaagttaaaatgttttatgactctctgttctgctgacaggcagagagtcacattgtgtaatttaatttcagtcagtcaatagatggcatccctcatcagggttgccagatggtgataacagtgtaaggccttgggtctaaggcatccacgactggaagggactactgatgttctgtgatacatcaggtttcagcacacaacttacatttctttgcctccaaattgaggcatttattatgatgttcatactttccctcttgtttgaaagtttctaattattaaatggtgtcggaattgttgtattttccttaggaattcagtggaacttatcttcattaaatttagctggtaccaggttgatatgacttgtcaatattatggtcaactttaagtcttagttttcgtttgtgcctttgattaataagtataactcttatacaataaatactgctttcctctaaaaagatcgtgtttaaattaacttgtagaaaatctgctggaatggttgttgttttccactgagaaagctaagccctacatttctattcagagtactgtttttagatgtgaaatataagcctgcggccttaactctgtattaaaaaaaatgtttttgtttaaaaaaaactgttcccataggtgcagcaaaccaccatggcacatgtatacctatgtaacaaacctgcacattttg

SEQ ID NO: 2 - protein, 245 aa, Homo sapiens
MASPSRRLQTKPVITCFKSVLLIYTFIFWITGVILLAVGIWGKVSLENYFSLLNEKATNVPFVLIATGTVIILLGTFGCFATCRASAWMLKLYAMFLTLVFLVELVAAIVGFVFRHEIKNSFKNNYEKALKQYNSTGDYRSHAVDKIQNTLHCCGVTDYRDWTDTNYYSEKGFPKSCCKLEDCTPQRDADKVNNEGCFIKVMTIIESEMGVVAGISFGVACFQLIGIFLAYCLSRAITNNQYEIV

SEQ ID NO: 3 - protein, 95 aa, Homo sapiens
RHEIKNSFKNNYEKALKQYNSTGDYRSHAVDKIQNTLHCCGVTDYRDWTDTNYYSEKGFPKSCCKLEDCTPQRDADKVNNEGCFIKVMTIIESEM

SEQ ID NO: 4 - protein, 41 aa, Artificial Sequence
  Peptide derived from Rabies virus and coupled to a poly-Arginine stretch
YTIWMPENPRPGTPCDIFTNSRGKRASNGGGGRRRRRRRRR

SEQ ID NO: 5 - protein, 29 aa, Artificial Sequence
  Ligand
YTIWMPENPRPGTPCDIFTNSRGKRASNG

SEQ ID NO: 6 - DNA, 19 bp, Artificial
  shRNA against rat TSPAN6 mRNA
ttcatcttttggatcactg

SEQ ID NO: 7 - DNA, 19 bp, Artificial
  shRNA against rat TSPAN6 mRNA
cagacatgagattaagaac